(19) United States
(12) Patent Application Publication
(10) Pub. No.: US 2023/0257733 A1
(43) Pub. Date: Aug. 17, 2023

(54) METHOD FOR ISOLATING NUCLEIC ACID

(71) Applicant: BioEcho Life Sciences GmbH, Koeln (DE)

(72) Inventors: Markus Mueller, Dormagen (DE); Maximilian Weiter, Erftstadt (DE); Joshua Leon Weis, Koeln (DE); Tobias Schughart, Dormagen (DE); Andreas Goedderz, Dormagen (DE)

(21) Appl. No.: 18/014,960

(22) PCT Filed: Jul. 7, 2021
(86) PCT No.: PCT/EP2021/068826

§ 371 Date: Jan. 6, 2023

(30) Foreign Application Priority Data
Jul. 7, 2020 (LU) 101918

(51) Int. Cl. C12N 15/10; C12Q 1/70

(52) U.S. Cl. CPC C12N 15/101; C12Q 1/70; C12Q 2600/118

(57) ABSTRACT

The present invention refers to a method for isolating a nucleic acid, said method comprising: a) provision of a fluid test sample, which comprises i) a biological sample, ii) a chaotropic agent with a concentration of at least 1 M in the fluid test sample, and iii) a detergent, b) contacting said fluid test sample with a medium for size-exclusion chromatography, and c) purifying the nucleic acid with size- exclusion chromatography. The present invention further relates to the use of any of the methods according to the present invention for detecting a viral infection as well as to a method for detecting a viral infection. The present invention further relates to a kit-of-parts comprising a medium for size- exclusion and a size-exclusion chromatography device for isolating the nucleic acid of the fluid test sample.

### FIELD OF THE INVENTION

The present invention refers to a method for isolating a nucleic acid, said method comprising: a) provision of a fluid test sample, which comprises i) a biological sample, ii) a chaotropic agent with a concentration of at least 1 M in the fluid test sample, and iii) a detergent, b) contacting said fluid test sample with a medium for size-exclusion chromatography, and c) purifying the nucleic acid with size-exclusion chromatography. The present invention further relates to the use of any of the methods according to the present invention for detecting a viral infection as well as to a method for detecting a viral infection. The present invention further relates to a kit-of-parts comprising a medium for size-exclusion and a size-exclusion chromatography device for isolating the nucleic acid of the fluid test sample.

### BACKGROUND OF THE INVENTION

The COVID-19 pandemic has resulted in an increased need for diagnostic testing, wherein PCR, RT-PCR and quantitative real-time polymerase chain reaction (q-RT-PCR) as well as Next Generation Sequencing (NGS) is essential for the identification of patients with coronavirus (SARS-CoV-2/ Covid-19). However, prior to any PCR-technique, the viral RNA must be isolated from samples. This exponential increase in demand for diagnostic testing has however resulted in a shortage of numerous reagents, in particular those associated with the lysis buffer required to extract the viral RNA, and has increased the need for reliable and time-saving techniques for isolation of viral RNA. Therefore, several challenges have to be considered. On the one hand, it is extremely important to achieve a reliable lysis of the viral particles, while on the other hand the sample gained after such a lysis should at the same time be essentially free of agents, e.g. from the lysis buffer itself, which have the potential or which may influence the PCR in a negative way, for example, by inhibition of components necessary for the performance of the PCR. Additionally, inhibitory activities that disturb the downstream analysis and originating from the biological sample should be excluded as far as possible.

Further, several methods of the state of the art apply very time consuming wash-bind-elute procedures, so that it is highly desired to get rid of these tedious and time-consuming steps. Especially, enzymatic lysis steps, which are generically part of common purification protocols are extremely time consuming and redundant with the methods described herein. The techniques known so far are not able to address these needs.

For example, Scallan et al. (Cork Institute of Technology, 2020, http://dx.doi.org/10.1101/2020.04.05.0264359) describes a lysis buffer for extraction of viral RNA.

Further, EP 04779084 (based on WO2005/012523) relates to a method for isolating small RNA molecules, inter alia, by adding an alcohol solution to a lysate. However, this method comprises the binding of the nucleic acid to a column, resulting in a tedious bind-wash-elute procedure.

Additionally, a time-consuming enzymatic lysis, optionally with an additional temperature incubation step is also quite common for the state of the art, which also results in loss of valuable time for diagnostic testing.

Thus, there is still a need for reliable, improved and alternative ways of isolating nucleic acid, especially viral RNA, so that not only high yield, high sensitivity and sustainability is given, but also the effective downstream performance of techniques, like PCR or RT-PCR, is guaranteed.

Accordingly, the technical problem underlying the present application is to provide a fast and reliable method of isolating nucleic acids, i.e. a method that addresses these needs described above.

### SUMMARY OF THE PRESENT INVENTION

The inventors of the present invention have surprisingly found that size-exclusion chromatography (SEC) enables to essentially get rid of inter alia chaotropic agents in one fast and simple step, thereby allowing the isolated nucleic acids to be employed in upstream processes, like sequencing and PCR-analysis.

Further, the inventors of the present invention unexpectedly found a method and conditions that allow tremendous time savings, e.g. by being able to omit several steps - compared to the conventional method used so far - for isolating and purifying nucleic acids, especially for isolating and purifying viral nucleic acid. Additional benefits are that the methods described herein in the description, the examples and claims may improve the quality and increase the amount of nucleic acids isolated from biological samples as illustrated herein. Moreover, the inventors of the present invention have found that a sufficient depletion of PCR inhibitor components, e.g. chaotropic agents, can be achieved with a SEC and which enables following downstream applications.

The present invention discloses an approach that enables the isolation of a nucleic acid, preferably for isolating a viral nucleic acid, more preferably a viral RNA, even more preferably a viral RNA of Coronaviridae, even more preferably a viral RNA of a SARS-CoV virus, most preferably the viral RNA of SARS-CoV-2, with high yield, speed, high sensitivity, sustainability, while being able to apply superior downstream performances in all following applications, like sequencing, PCT and NGS. Most importantly, the method of the present invention enables the isolation of a nucleic acid without any tedious bind-wash-elute procedure and allows depletion of substances, maybe used for lysis, which can have the potency to inhibit the performance of any PCT or RT-PCR, which is performed later on. This improved procedure speeds nucleic acid purification dramatically up and reduces the number of handling steps significantly. At the same time, it is not prone to deliver false -negative results due to co-elution of enzyme inhibitors, like chaotropic reagents and organic solvents, into the eluate to compromise or inhibit downstream analyses, like PCR or RT-PCR. This procedure is thus very time-saving and especially advantageous for the isolation of a viral RNA, preferably a viral RNA of Coronaviridae, more preferably a viral RNA of a SARS-CoV virus and most preferably the viral RNA of SARS-CoV-2.

Thus, herein is described a previously unknown, novel mechanism for isolating nucleic acid based on size exclusion chromatography materials, that allows a rapid buffer exchange (desalting) of a solution consisting of highly concentrated chaotropic salts and dissolved nucleic acids in an extremely fast and efficient manner, thus making the nucleic acids in the solution accessible for subsequent sequencing or inhibition-free PCR analysis.

The present invention relates to a method for isolating a nucleic acid, said method comprising:
a) provision of a fluid test sample, which comprises
   i) a biological sample,
   ii) a chaotropic agent with a concentration of at least 1 M in the fluid test sample, and
   iii) a detergent,
b) contacting said fluid test sample with a medium for size-exclusion chromatography, and
c) purifying the nucleic acid with size-exclusion chromatography.

In a preferred embodiment of the method of the present invention, the nucleic acid of step c) is directly applied to PCR, RT-PCR or NGS.

The method of the present invention is preferably conducted without the addition of a protease.

The method of the present invention is preferably conducted without a bind-wash-elute-step.

In a preferred embodiment of the method of the present invention, the chaotropic agent has a concentration of at least 1.5 M in the fluid test sample, preferably of at least 2 M in the fluid test sample, more preferably of at least 2.5 M in the fluid test sample, even more preferably of at least 3 M in the fluid test sample, even more preferably of at least 3.5 M in the fluid test sample.

In a preferred embodiment of the method of the present invention, the medium for size exclusion is a resin used for size exclusion chromatography (SEC); preferably a hydroxylated methacrylic polymer, a cross-linked dextrane or a cross-linked agarose; more preferably a dextrane cross-linked with N,N′-methylenebisacrylamide; a water-based mobile phase, such as water, an aqueous organic solvent or an aqueous buffer/ solution mobile phase.

For the method of the present invention, it is preferred that the chaotropic agent is guanidinium thiocyanate or guanidinium hydrochloride, more preferably guanidinium thiocyanate.

In a preferred embodiment of the method of the present invention, the detergent is a non-ionic detergent, preferably Triton, more preferably Triton X-100, or the detergent is a salt of lauroyl sarcosinate, preferably sodium lauroyl sarcosinate, or a derivative thereof. Even more preferably, the detergent is Triton X-100. Also even more preferably, the detergent is sodium lauroyl sarcosinate.

For the method of the present invention, it is preferred that said nucleic acid is RNA and/or DNA, preferably RNA.

It is preferred for the method of the present invention that said RNA is a viral RNA, more preferably a viral RNA of Coronaviridae, even more preferably a viral RNA of a SARS-CoV virus and most preferably the viral RNA of SARS-CoV-2.

It is preferred for the method of the present invention that the biological sample is a viral sample, a fecal sample, a saliva sample, a sputum sample, a mouth swab sample, a throat swab sample or a nasal swab sample.

In a preferred embodiment of the method of the present invention, said fluid test sample further comprises EDTA, Triton X-100 DTT, citrate monohydrate, dihydro sodium citrate, or a buffering substance, more preferably Tris-HCI. In a preferred embodiment of the method of the present invention, said fluid test sample further comprises a reducing agent, preferably DTT, TCEP or a derivative thereof.

It is also preferred for the method of the present invention, that the method further comprises a step of heating the fluid test sample, preferably at a temperature in the range from about 80° C. to about 95° C., preferably before step b). It is further preferred that said heating of the fluid test sample is conducted at a temperature in the range from about 80° C. to about 95° C. for about 5 to 15 minutes, more preferably for about 10 minutes.

For the method of the present invention, it is preferred that said provision of a fluid test sample comprises the step of contacting a viral sample with a lysis buffer.

The present invention further relates to the use of any of the methods as described herein for detecting a viral infection, more preferably for detecting a viral nucleic acid, even more preferably a viral RNA, even more preferably a viral RNA of Coronaviridae, even more preferably a viral RNA of a SARS-CoV virus and most preferably the viral RNA of SARS-CoV-2. Thus, the present invention is able to detect any viral infection in general by the methods and the use as described herein.

The present invention also refers to a kit-of-parts comprising a medium for size-exclusion and a size-exclusion chromatography device for isolating a nucleic acid of a fluid test sample, wherein the fluid test sample comprises i) a biological sample, ii) a chaotropic agent with a concentration of at least 1 M in the fluid test sample, and iii) a detergent.

The present invention also relates to a method for detecting a viral infection, preferably for detecting a viral nucleic acid, more preferably a viral RNA, even more preferably a viral RNA of Coronaviridae, even more preferably a viral RNA of a SARS-CoV virus, most preferably the viral RNA of SARS-CoV-2, wherein said method comprises:
a) provision of a fluid test sample, which comprises
   i) a biological sample,
   ii) a chaotropic agent with a concentration of at least 1 M in the fluid test sample, and
   iii) a detergent,
b) contacting said fluid test sample with a medium for size-exclusion chromatography, and
c) purifying the nucleic acid with size-exclusion chromatography.

### BRIEF DESCRIPTION OF THE FIGURES

FIG. 1 shows the experimental arrangement for the plate 1 used in Example 1 and shows the respective volume of the fluid test sample, the resin-volume and the respective column applied.

FIG. 2 shows the experimental arrangement for the plate 2 used in Example 1 and shows the respective volume of the fluid test sample, the resin-volume and the respective column applied.

FIG. 3 shows the results gained from the inhibition-PCR of the eluates from plate 2 of Example 1.

FIG. 4 shows the results with regard to the samples used in experiment 2 of Example 7 and shows the respective PCR spikes and Ct-values.

FIG. 5 shows the Ct-values gained for experiment 3 of Example 7 for various different concentrations of GITC and GHCI, different resin volumes and column materials.

FIG. 6 shows the Ct-values gained for experiment 4 of Example 7 for various different concentrations of GITC, 800 µl resin volume and column S400.

FIG. 7 shows the procedure for establishing a standard-curve for the experiments of Example 7.

FIG. 8 shows a detail of the procedure for establishing a standard-curve for the experiments of Example 7.

FIG. 9 shows the particle size distribution and exclusion limits of filter materials used in Example 9 as described herein.

FIG. 10 shows the experimental setup of the experiment according to Example 9 as described herein.

FIG. 11 shows the serial dilution of GuSCN and corresponding conductivity concerning Example 9 as described herein.

FIG. 12 shows the calculated GuSCN-concentrations in the eluates concerning Example 9 as described herein.

FIG. 13 shows the Ct-values obtained in Experiment 10 as described herein.

FIG. 14 shows the list of filter materials used in Example 11 as described herein.

FIG. 15 shows the Ct-values obtained in Experiment 11 as described herein.

FIG. 16 shows the Ct-values obtained in Experiment 12 as described herein.

### DETAILED DESCRIPTION OF THE PRESENT INVENTION

### Definitions

As used herein, the term “nucleic acid” comprises any type of DNA or RNA as well as a mixture of DNA and RNA of any type.

Being widely used for the isolation and separation of proteins and protein mixtures, size exclusion chromatography (SEC) filter materials are known to the person skilled in the art. Their principle is based on the separation of molecules according to their size. It is possible to separate very large and very small molecules from each other (group separation) or several large molecules that only differ from each other in molecular size (high-resolution fractionation). Usually, chromatography columns are filled with the filter material and the analyte mixture to be separated is processed through the column with a mobile phase. The main factors influencing the retention time of the analytes in the column are the particle and pore size of the filter materials used and the flow rate of the mobile phase through the column. Normally, the exact pore size of the filter materials is not given, instead a size exclusion limit in the unit Dalton [Da] is provided. Especially for the separation of large biomolecules (e.g. proteins or nucleic acids) from small molecules (e.g. salts, metabolites, dyes), the manufacturers of SEC filter materials recommend materials with the smallest possible pore sizes (e.g. Cytiva Sephadex resins). The separation of large biomolecules (usually mixtures of proteins) from each other, on the other hand, is more likely to be achieved by using large-pored filter materials (e.g. Cytiva Superdex, Superose and Sephacryl resins). Thus, as used herein, the term “size-exclusion chromatography”, also known as molecular sieve chromatography, means any chromatographic method, in which molecules in solution are separated by their size, and in some cases molecular weight. As used herein, “positive chromatography” herein refers to a method of enriching a compound by retaining the compound to be enriched in a chromatography device, wherein undesired contaminants, inhibitors and other components are washed away and the compound to be enriched is eluted in a final step. “Negative chromatography” herein refers to a method of enriching a compound by retaining the undesired contaminants in a chromatography device and/or a resin, while the compound to be enriched passes the chromatography device.

As used herein, the term “non-nucleic acid components” comprises all non-nucleic acid compounds in a solution, especially those that compromise or even inhibit subsequent or downstream applications like PCR, cloning, ligation and/or sequencing of nucleic acids. Especially comprised by the term “non-nucleic acid components” are proteins, salts, chaotropic agents, detergents, organic or inorganic solvents, dyes, metabolites, sample debris, low molecular molecules (e.g. nucleotides etc.) and/or PCR inhibitors.

As used herein, the term “resin” comprises an insoluble matrix or medium capable of interacting with binding partners. Typically, a resin is used in a chromatographic procedure, wherein the resin retains different components depending on their characteristics to a different extent and thereby separates the different components of the solution or mixture.

A “biological sample” as used herein, refers to any biological material containing nucleic acids, preferably RNA, more preferably a viral nucleic acid, even more preferably a viral RNA, even more preferably a viral RNA of Coronaviridae, even more preferably a viral RNA of a SARS-CoV virus, and most preferably the viral RNA of SARS-CoV-2. In one embodiment, biological samples comprise cells and/or cell-free nucleic acids from gram-positive or gram-negative bacteria, virus, protozoa, chromista, fungi, plants and/or animals. In another embodiment, the biological samples are isolated from fungi, plants and/or animals, but may contain biological samples consisting of cells from bacteria, protozoa, chromista, fungi, plants and/or animals. In one embodiment, animal refers to vertebrates, preferably tetrapods, fish, and/or birds, more preferably mammals and even more preferably cows, cats, dogs, horses, pigs, humans. In another preferred embodiment of the invention, animals refer to animals for production/ livestock. In an additional embodiment of the invention, the biological sample refers to a forensic case sample. A viral RNA means that a virus comprises RNA as genetic material. This nucleic acid of a viral RNA is usually single-stranded RNA (ssRNA), but may be double-stranded RNA (dsRNA). Notable human diseases caused by RNA viruses include the common cold, influenza, SARS, COVID-19, hepatitis C, hepatitis E, West Nile fever, Ebola virus disease, rabies, polio and measles. A viral DNA means that a virus comprises DNA as genetic material.

A “chaotropic agent” is a molecule in water solution that can disrupt the hydrogen bonding network between water molecules (i.e. exerts chaotropic activity). This has an effect on the stability of the native state of other molecules in the solution, mainly macromolecules (proteins, nucleic acids) by weakening the hydrophobic effect. For example, a chaotropic agent reduces the amount of order in the structure of a protein formed by water molecules, both in the bulk and the hydration shells around hydrophobic amino acids, and may cause its denaturation. Conversely, an anti-chaotropic agent (kosmotropic) is a molecule in an aqueous solution that will increase the hydrophobic effects within the solution. Chaotrophic agents or salts, for example guanidinium thiocyanate/ GuSCN, are very widely used in molecular biology. They are, for example, utilized for the inactivation of viruses or for the lysis of cells and tissues. Currently chaotropic agents/ salts have a dual function in state of the art silica-based nucleic acid extractions. Not only they fulfil a function in the lysis of biological material (usually in combination with a protease), but also lead to a binding of the released nucleic acids to the silica surface in combination with alcohols. Since the nucleic acids obtained are mostly used downstream in PCR assays, it is very important that they do not contain PCR inhibitors. Because chaotropic agents/ salts are strong PCR inhibitors, the chaotropic salts must be rinsed away by repeated washing with pure ethanol. At the end of the silica extraction, the bound nucleic acids are detached and eluted in an aqueous buffer. The main disadvantage of silica-based nucleic acid extractions is thus the need for multiple washing of the bound nucleic acids before they can be eluted in aqueous buffer. The chaotropic agent used within the present invention is able to lyse the respective biological sample, without the use of an enzymatic digestion for lysis and without the need to apply a further/ additional temperature step or time consuming multiple washing steps.

The term “detergent”, as used within the context of the present invention, may mean a blended product that contains (a) surfactant(s) plus other ingredients (typically called builders) to make a formulated detergent. By naming convention in the detergent industry, a formulated detergent is classified by the charge on the surfactant that is present in the detergent. You can also have formulated detergents that are anionic, cationic, non-ionic, amphoteric and zwitter-ionic. These formulated detergents can contain builders such as sodium phosphates, sodium silicates, sodium carbonates, potassium hydroxide, citric acid and many other ionic salts or acids. Many anionic surfactants have sodium or potassium metal ions present in their salt form when found in the detergent. One example of a non-ionic detergent, being effective for lysis and which can be used in the method of the present invention, but is not limited thereto, is Triton, preferably Triton X-100. Further, an example of an anionic detergent, being effective for lysis and which can be used in the method of the present invention, but is not limited thereto, is a salt of lauroyl sarcosinate, preferably sodium lauroyl sarcosinate. Sodium lauroyl sarcosinate has the advantage that it is, for example, highly soluble together with chaotropic agents/ salts.

The “eluate”, as used in the context of the present invention, is the product of applying the provided fluid test sample to step b) of the methods of the present invention as defined herein and step c) of purifying the nucleic acid with size-exclusion chromatography. Thus, the provided fluid test sample is contacted with a medium for size-exclusion chromatography and the size-exclusion chromatography is performed. The product of this procedure is the so called eluate, which may be collected in the method of the present invention. Thus, a negative chromatography as defined above is applied. The term “contacting” may mean to bring the fluid test sample into any form of contact with the medium for size-exclusion chromatography, for example, in a column for a certain time or time range. The eluate may then be, for example, subjected to further steps, e.g. for increasing the concentration of the gained nucleic acid in the eluate, before it may be directly applied to PCR, RT-PCR or NGS afterwards.

“Polymerase chain reaction” (PCR), as used within the context of the present invention, is the technique in molecular biology, wherein a DNA polymerase is used to amplify a DNA fragment by enzymatic replication in vitro. As the PCR progresses, the generated DNA itself (the amplicon) is used as a template for replication. This sets in motion a chain reaction, in which the DNA template is exponentially amplified. With PCR, it is possible to amplify one or more copies of a DNA fragment by several orders of magnitude, generating millions or more copies of the DNA fragment. PCR employs a thermostable polymerase, dNTP, and a pair of primers. PCR is conceptually divided into 3 reactions, each of which is typically assumed to occur over time at each of three temperatures (denaturation, hybridization, and extension that occur at 3 temperatures for 3 time periods each cycle). A “real-time polymerase chain reaction” (real-time PCR or RT-PCR or rt-PCR), also known as quantitative polymerase chain reaction (qPCR), is based on the classical polymerase chain reaction (PCR). It monitors the amplification of a targeted DNA/ RNA molecule during the PCR (i.e., in real time), not at its end, as in the conventional PCR. Real-time PCR can be used quantitatively (quantitative real-time PCR) and semi-quantitatively (i.e., above/ below a certain amount of RNA/ DNA molecules) (semiquantitative real-time PCR).

### DETAILED DESCRIPTION OF THE EMBODIMENTS

The present invention relates to a method for isolating a nucleic acid, said method comprising:
a) provision of a fluid test sample, which comprises
   i) a biological sample,
   ii) a chaotropic agent with a concentration of at least 1 M in the fluid test sample, and
   iii) a detergent,
b) contacting said fluid test sample with a medium for size-exclusion chromatography, and
c) purifying the nucleic acid with size-exclusion chromatography.

Certain different embodiments including preferred embodiments of the invention will be described in the following detailed description of the invention. Although all nucleic acids are envisaged by this invention, the purification of RNA, especially viral RNA, is preferred.

In preferred embodiments, the biological sample is a body fluid sample, stool sample, an environmental sample, a cell culture sample, a bone marrow sample, a sewage sample, a food sample, a milk sample, a forensic sample, a biological molecule production sample, a protein preparation sample, a lipid preparation sample, a carbohydrate preparation sample, and any combination thereof, wherein, optionally, the body fluid sample is one of a blood sample, a serum sample, an amniotic fluid sample, a semen sample, a lymphatic fluid sample, a cerebrospinal fluid sample, a nasopharyngeal wash sample, a sputum sample, a mouth swab sample, a throat swab sample, a nasal swab sample, a bronchoalveolar lavage sample, a bronchial secretion sample, and an urine sample. More preferred, the biological sample is a viral sample, a fecal sample, a saliva sample, a sputum sample, a mouth swab sample, a throat swab sample or a nasal swab sample (nasopharyngeal SWABs). Any of these mentioned samples may be provided in a dry form, being within a chaotropic transport buffer or a non-chaotrophic media (e.g. COPAN UTM, COPAN eSWAB) or is not provided in a non-chaotropic transport buffer. In any way, in the methods of the present invention, the nucleic acid, which should be isolated, is from the biological sample, meaning that the biological sample contains the nucleic acid to be isolated in any form.

For the method of the present invention, it is preferred that the chaotropic agent is guanidinium thiocyanate or guanidinium hydrochloride, more preferably guanidinium thiocyanate. “Guanidinium thiocyanate” is generally used synonymously with “guanidinium isothiocyanate”, “guanidine thiocyanate” or “GITC”. “Guanidinium hydrochloride” is generally used synonymously with “guanidinium chloride”, “GuHCl”, “GHCl”, “GdnHCl” or “GdmCl”. In one further embodiment of the present invention, the chaotropic agent is perchlorate or urea. For the method of the present invention, the chaotropic agent has a concentration of at least 1 M in the fluid test sample. It is preferred for the method of the present invention, that the chaotropic agent has a concentration of at least 1.5 M in the fluid test sample. It is more preferred for the method of the present invention, that the chaotropic agent has a concentration of at least 2 M in the fluid test sample. It is even more preferred for the method of the present invention, that the chaotropic agent has a concentration of at least 2.5 M in the fluid test sample. It is even more preferred for the method of the present invention, that the chaotropic agent has a concentration of at least 3 M in the fluid test sample. It is even more preferred for the method of the present invention, that the chaotropic agent has a concentration of at least 3.5 M in the fluid test sample. It is even more preferred for the method of the present invention, that the chaotropic agent has a concentration of at least 4 M in the fluid test sample.

Various agents suitable for biological sample lysis are available, wherein an important feature of those agents useful in lysis of a biological sample for nucleic acid purification is the capability to lyse cells and as an optional feature to inhibit the activity of nucleases. Another important factor of an efficient lysis is generally the incubation time. However, the inventors of the present invention have found that with the method of the present invention no separate or time-consuming incubation step is necessary. This means that already after a very short time-range, e.g. of 10 seconds, the fluid test sample can be applied to the further steps b) and c) of the methods of the present invention. Further, no enzymatic lysis step or heating step has to be applied. If viral particle are present, just the contact thereof with the chaotropic agent and the detergent leads to lysis.

Further, the fluid test sample comprises a detergent.

It is preferred in one embodiment of the method of the present invention, that the detergent is a non-ionic detergent. Non-ionic detergents are characterized by their uncharged, hydrophilic headgroups. Typical non-ionic detergents are based on polyoxyethylene or a glycoside. Common examples thereof include Tween, Triton, and the Brij series. These materials are also known as ethoxylates or PEGylates and their metabolites, nonylphenol. Glycosides have a sugar as their uncharged hydrophilic headgroup. Examples include octyl thioglucoside and maltosides. HEGA and MEGA series detergents are similar, possessing a sugar alcohol as headgroup.

In one embodiment of the method of the present invention, it is preferred that the detergent is a non-ionic detergent selected from the group consisting of Triton X-100, Triton X-114, NP-40, Brij-35, Brij-58, Tween 20, Tween 80, octyl glucoside and octyl thioglucoside. More preferably, the non-ionic detergent used in the method of the present invention is Triton, even more preferably Triton X-100. Thus, in a more preferred embodiment, the detergent used for the lysis of the biological sample is Triton, even more preferably Triton X-100.

It is also preferred in one embodiment of the method of the present invention, that the detergent is an anionic detergent, more preferably a salt of lauroyl sarcosinate, even more preferably sodium lauroyl sarcosinate. Typical anionic detergents are alkylbenzenesulfonates. The alkylbenzene portion of these anions is lipophilic and the sulfonate is hydrophilic. Two different varieties have been popularized, those with branched alkyl groups and those with linear alkyl groups.

In one preferred embodiment of the method of the present invention, the anionic detergent used in the method of the present invention is an acyl sarcosinate. The acyl sarcosines (e.g., cocoyl sarcosine, lauroyl sarcosine, myristoyl sarcosine, oleoyl sarcosine, stearoyl sarcosine) are modified fatty acids, and acyl sarcosinates (e.g., sodium cocoyl sarcosinate, sodium lauroyl sarcosinate, sodium myristoyl sarcosinate, ammonium cocoyl sarcosinate, ammonium lauroyl sarcosinate) are their respective salts. Another term for acyl is alcanoyl, which can be used interchangeably herein. Such an acyl sarcosinate is based on sarcosinate, wherein the hydrogen attached to the nitrogen of the sarcosinate is substituted with an acyl/ alcanoyl residue/ group, which can be, for example, branched or un-branched, with a double bond or without a double-bond. For example, an acyl/ alcanoyl as used herein can be, without being limited to it, a stearoyl, a cocoyl, a lauroyl, a myristoyl or an oleyl residue/ group.

Thus, further examples of an anionic detergent, which can be used in the method of the present invention, without being limited to it, are cocoyl sarcosine, lauroyl sarcosine, myristoyl sarcosine, oleoyl sarcosine, stearoyl sarcosine, sodium cocoyl sarcosinate, sodium lauroyl sarcosinate, sodium myristoyl sarcosinate, ammonium cocoyl sarcosinate, ammonium lauroyl sarcosinate, and salts or derivatives thereof.

In one preferred embodiment of the method of the present invention, the anionic detergent is a salt of lauroyl sarcosinate, even more preferably sodium lauroyl sarcosinate. Sodium lauroyl sarcosinate is highly soluble in chaotropic salts/ agents. Thus, in a more preferred embodiment of the method of the present invention, the detergent used for the lysis of the biological sample is a salt of lauroyl sarcosinate, even more preferably sodium lauroyl sarcosinate. In one preferred embodiment of the method of the present invention, the anionic detergent is not sodium dodecyl sulfate (SDS).

In one embodiment of the method of the present invention, it is preferred that the detergent is a non-ionic detergent, preferably Triton, more preferably Triton X-100, or a salt of lauroyl sarcosinate, more preferably sodium lauroyl sarcosinate, and derivates thereof.

Triton X-100 is a non-ionic detergent having a hydrophilic polyethylene oxide chain and an aromatic hydrocarbon lipophilic or hydrophobic group. Other names of Triton X-100 are polyethylene glycol, TX-100, Mono 30 or octyl phenol ethoxylate.

Sodium lauroyl sarcosinate is highly soluble together with chaotropic agents/ salts. It is also known as sarkosyl and is amphiphilic due to the hydrophobic 12-carbon chain (lauroyl) and the hydrophilic carboxylate.

In one embodiment of the method of the present invention, the detergent is a zwitter-ionic detergent, preferably 3-[(3-cholamidopropyl)dimethylammonio]-1-propanesulfonate (CHAPS) or 3-[(3-cholamidopropyl)dimethylammonio]-2-hydroxy-1-propanesulfonate (CHAPSO).

In one embodiment of the method of the present invention, the concentration of the detergent is at least about 1% (v/v). In yet another embodiment of the method of the present invention, the concentration of the detergent is at least about 1.5% (v/v), at least about 2% (v/v), at least about 3% (v/v), at least about 4% (v/v), at least about 5% (v/v), at least about 6% (v/v), at least about 7% (v/v) or at least about 8% (v/v). In a preferred embodiment of the method of the present invention, the concentration of the detergent is at least about 0.5% (v/v). In a more preferred embodiment of the method of the present invention, the concentration of the detergent is in the range from about 0.5% (v/v) to about 10% (v/v). In a more preferred embodiment of the method of the present invention, the concentration of the detergent is in the range from about 1% (v/v) to about 8% (v/v). In an even more preferred embodiment of the method of the present invention, the concentration of the detergent is in the range from about 2% (v/v) to about 7% (v/v). In an even more preferred embodiment of the method of the present invention, the concentration of the detergent is in the range from about 3% (v/v) to about 7% (v/v). In an even more preferred embodiment of the method of the present invention, the concentration of the detergent is in the range from about 3% (v/v) to about 6.5% (v/v). In an even more preferred embodiment of the method of the present invention, the concentration of the detergent is in the range from about 4% (v/v) to about 6.5% (v/v). In an even more preferred embodiment of the method of the present invention, the concentration of the detergent is in the range from about 5% (v/v) to about 6.5% (v/v). In an even more preferred embodiment of the method of the present invention, the concentration of the detergent is in the range from about 5.5% (v/v) to about 6.5% (v/v). In an even more preferred embodiment of the method of the present invention, the concentration of the detergent is about 6% (v/v).

In one preferred embodiment of the method of the present invention, the method further comprises a step of heating the fluid test sample. It is further preferred that said step of heating the fluid test sample is at a temperature in the range from about 80° C. to about 95° C., more preferably at a temperature in the range from about 85° C. to about 95° C., even more preferably at a temperature in the range from about 90° C. to about 95° C., and even more preferably at a temperature of about 95° C. It is further preferred that said step of heating the fluid test sample is conducted before step b) of the method of the present invention. Said step of heating the fluid test sample may be conducted after contacting the biological sample with the lysis buffer. Said step of heating the fluid test sample may be comprised in step a) of the method of the present invention. Thus, step a) of the method of the present invention, the provision of the fluid test sample, may comprise the step of heating the fluid test sample, preferably at a temperature in the range from about 80° C. to about 95° C., more preferably at a temperature in the range from about 85° C. to about 95° C., even more preferably at a temperature in the range from about 90° C. to about 95° C., and even more preferably at a temperature of about 95° C. Said step of heating the fluid test sample is preferably carried out for about 5 to about 15 minutes, more preferably for about 8 to about 12 minutes and even more preferably for about 10 minutes.

For the method of the present invention, an “eluate” may be the product gained after contacting the provided fluid test sample with a medium for size exclusion chromatography in step b) and after performing the size exclusion chromatography. Thus, step b) of the present invention may comprise contacting said fluid sample with a medium for size-exclusion chromatography. Step c) of the present invention, purifying the nucleic acid with size-exclusion chromatography, may comprise the step of collecting the eluate. In one preferred embodiment of the method of the present invention, contacting said fluid test sample with a medium for size-exclusion chromatography is performed in a column. In one preferred embodiment of the method of the present invention, contacting said fluid test sample with a medium for size-exclusion chromatography is performed in a column for a predetermined time range. In one preferred embodiment of the method of the present invention, contacting said fluid test sample with a medium for size-exclusion chromatography is performed in a column for 10 seconds to 20 min. In one more preferred embodiment of the method of the present invention, contacting said fluid test sample with a medium for size-exclusion chromatography is performed in a column for 10 seconds to 10 min. In one even more preferred embodiment of the method of the present invention, contacting said fluid test sample with a medium for size-exclusion chromatography is performed in a column for 10 seconds to 7 min. In one even more preferred embodiment of the method of the present invention, contacting said fluid test sample with a medium for size-exclusion chromatography is performed in a column for 10 seconds to 5 min. In one even more preferred embodiment of the method of the present invention, contacting said fluid test sample with a medium for size-exclusion chromatography is performed in a column for 20 seconds to 5 min. In one even more preferred embodiment of the method of the present invention, contacting said fluid test sample with a medium for size-exclusion chromatography is performed in a column for 10 seconds to 3 min.

In a preferred embodiment of the method of the present invention, the nucleic acid of step c) is directly applied to PCR, RT-PCR or NGS. For the PCR, the RT-PCR or the NGS, it is preferred that the polymerase used for PCR, RT-PCR or NGS is selected from the group consisting of Taq-polymerase, Tfl-polymerase, Tma-polymerase, Tne-polymerase, Tth-polymerase, Pfu-polymerase, Pwo-polymerase, KOD-polymerase, Tli-polymerase, Tag-polymerase, Tce-polymerase, Tgo-Polymerase, TNA1-polymerase, Tpe-polymerase, Tthi-polymerase, Neq-polymerase, Pab-polymerase, T4-DNA-polymerase, T6-DNA-polymerase and T7-DNA-polymerase. More preferably, the polymerase used for PCR, RT-PCR or NGS is selected from the group consisting of Taq-polymerase, Tag-polymerase and Tgo-Polymerase. Most preferably, the polymerase used for PCR, RT-PCR or NGS is a Taq-polymerase.

The method of the present invention is preferably conducted without the addition of a protease. Such a protease(s) is/are may be, for example, a protease from Bacillus licheniformis, a protease from Bacillus spec., a protease from Staphylococcus aureus, a protease from Bacillus amyloliquefaciens, a protease from Coprinus spec. or a protease from Aspergillus oryzae.

In a preferred embodiment of the method of the present invention, the method is conducted without a bind-wash-elute-step. This allows to safe a tedious step being one of the most time-consuming steps. The time-advantage is extremely important, when it is necessary to conduct several times the method of the present invention within a short time limit or range, for example, for detecting the viral RNA or DNA in a pandemic situation.

In a preferred embodiment of the method of the present invention, the chaotropic agent has a concentration of at least 1.5 M in the fluid test sample, more preferably of at least 2 M in the fluid test sample, more preferably of at least 2.5 M in the fluid test sample, even more preferably of at least 3 M in the fluid test sample, even more preferably of at least 3.5 M in the fluid test sample and even more preferably of at least 4 M in the fluid test sample.

In a preferred embodiment of the method of the present invention, the medium for size exclusion chromatography is a resin used for size exclusion chromatography (SEC). SEC is a chromatographic method, wherein molecules are separated based on their size, or more precisely based on their hydrodynamic volume. Preferably, such a resin for size exclusion chromatography may be a hydroxylated methacrylic polymer or a cross-linked dextrane, preferably a dextrane cross-linked with N,N′-methylenebisacrylamide. Preferably, such a resin for size-exclusion chromatography may also have a water-based mobile phase, such as water, an aqueous organic solvent or an aqueous buffer/solution mobile phase. Commonly, a solid matrix is able to form a gel bed, when suspended in an aqueous medium. Components of such a solid matrix comprise Sephadex, Sephacryl, hydroxylated methacrylic polymers, crosslinked agarose, silica-based materials, diatomaceous earth, polystyrene/divinyl benzene, and/or ceramic hydroxy apatite. One or more components may also be mixed. The one or more component is suspended in a buffer and packed in the hollow body of a column. Columns may be made of glass, plastic, Teflon or any other material that neither reacts with the mobile phase nor the analyte. The bead or amorphous particle size of such a medium or resin (material) can range from 1 µm to 500 µm, preferably 25 µm to 400 µm. The average diameters of such a material or resin may depend on volume and the debris concentration loaded onto the respective resin or column beds.

In a more preferred embodiment of the method of the present invention, the medium of the size-exclusion chromatography is a resin selected from the group consisting of Sephacryls, preferably Sephacryl 100, Sephacryl 200, Sephacryl 300, Sephacryl 400 or Sephacryl 500, more preferably Sephacryl 400; Toyopearls, preferably Toyopearl HW 65 S, Toyopearl HW 65 F or Toyopearl HW 65 C, and a SEC resin comprising cross-linked agarose, like WorkBeads from Bioworks, e.g. WorkBeads 40/10000, 40/1000 and 40/100.

The step b), contacting said fluid test sample with a medium for size-exclusion chromatography, can be carried out at a temperature in the range of about 10° C. to about 60° C. In a preferred embodiment, step b) is carried out at a temperature in the range of about 15° C. to 40° C. In a more preferred embodiment, step b) is carried out at a temperature in the range of about 18° C. to about 28° C. In an even more preferred embodiment, step b) is carried out at a temperature in the range of about 20° C. to about 25° C., e.g. at room temperature.

The fluid test sample to be purified is then applied to the gel’s bed upper surface, and allowed to pass through the gel, e.g. forced by centrifugation, vacuum or pressure. Within this invention, preferably centrifugal forces are applied to move the mobile phase down the column, wherein the columns are spun in a centrifuge (so-called spin column technique, “centrifugation column”). Due to the nature of the resin, pores of a certain size exist inside the gel. Small molecules are able to penetrate the pores, and therefore move through the resin more slowly, being retained as they pass down the column, while large molecules cannot penetrate the pores and move down the column more quickly. After having passed the column, the mobile phase (now referred to as “eluate”), containing the purified nucleic acid, is then collected at the outlet of the column. To retain the resin within the hollow body of the column, a porous frit, filter, fleece or membrane is preferably placed between the outlet of the column and the solid matrix, wherein nucleic acids of all sizes may pass said frit, filter, fleece or membrane.

In SEC, the size exclusion limit defines the molecular weight or length of a nucleic acid, where molecules are too large to be trapped in the stationary phase/ the resin. The size-exclusion limit of a resin is defined by the composition of the resin and can be influenced by particle size, the type of resin and the degree of crosslinking. In one embodiment of the invention, the size exclusion limit of the resin is between 1 and 10⁶ base pairs (bp). In a preferred embodiment, the size exclusion limit is between 5 and 10000 bp and in a more preferred embodiment, the size exclusion limit is in the range of 20 to 2000 bp. As used herein, the units “base pairs” (bp) and “nucleotides” (nt) can be used interchangeably.

The resin is preferably incorporated into a column. This column comprises a hollow body having an inlet and an outlet, the hollow body comprising a solid matrix providing size excluding properties. Preferably, it additionally comprises a porous frit, filter, fleece or membrane, preferably allowing nucleic acids of any size to pass, placed between the outlet and the resin to retain the resin within the column. The column optionally comprises a non-porous ring placed between the porous frit, filter, fleece or membrane and the resin, sealing the outer area of the frit, filter, fleece or membrane, to prevent the mobile phase from entering the frit without passing the resin. Also, optionally, the column comprises at least one removable closing device to seal the inlet and/or the outlet of the chromatographic unit. Further optionally, the column comprises at least one collection tube to collect the mobile phase (eluate) after having passed the resin. The material of the column may be selected from the group consisting of glass, polypropylene, polycarbonate or polyethylene.

Preferably, in one embodiment of the method of the present invention, well plates are used for step b) and/ or step c) of the method of the present invention. Especially, well plates or microplates of the type 96-well- or 384-well-microtiter-plates are used. For example, polystyrene multititer-plates for immunoassay and high throughput screening applications may be used.

In one embodiment of the method of the present invention, wherein a centrifugal force is used to facilitate collecting the eluate of the size-exclusion chromatography, e.g. out of a resin, the centrifugation step is executed at 400 g to 3000 g for about 0.5 to about 5 min. This slow centrifugation increases the quality and amount of isolated nucleic acids, preferably RNA. Preferably, the centrifugation step is executed at 400 g to 3000 g. More preferably, the centrifugation step is executed at 400 g to 3000 g for about 0.5 min to about 5 min, even more preferably for about 1 min at 1000 x g.

To decrease the volume of the eluate, thereby increasing the concentration of the nucleic acids in the eluate, it is possible to apply a high centrifugal force to the resin, herein referred to as “preconditioning”. In a preferred embodiment, the resin is centrifuged at least 1 min at at least 300 g for collecting the eluate. In a more preferred embodiment, the resin is centrifuged at least 2 min at at least 300 g for collecting the eluate.

In a preferred embodiment of the method of the present invention, said nucleic acid is RNA and/or DNA, preferably RNA.

It is preferred for the method of the present invention that said RNA is a viral RNA, more preferably a viral RNA of Coronaviridae, even more preferably a viral RNA of a SARS-CoV virus and most preferably the viral RNA of SARS-CoV-2. Coronaviridae is a family of enveloped, positive-sense, single-stranded RNA viruses, which infects amphibians, birds, and mammals. The viral genome is 26-32 kilobases in length. The particles are typically decorated with large (^{~}20 nm), club- or petal-shaped surface projections (the “peplomers” or “spikes”), which in electron micrographs of spherical particles create an image reminiscent of the solar corona.

In a preferred embodiment of the method of the present invention, said fluid test sample further comprises EDTA, Triton X-100, dithiothreitol (DTT), citrate monohydrate, dihydro sodium citrate, or a buffering substance, more preferably Tris-HCl or other aqueous buffers. In a preferred embodiment of the method of the present invention, said fluid test sample further comprises a reducing agent, more preferably dithiothreitol (DTT), TCEP or derivatives thereof. In a more preferred embodiment of the method of the present invention, said fluid test sample comprises a buffer comprising guanidinium thiocyanate, Triton X-100 and DTT. In a more preferred embodiment of the method of the present invention, said fluid test sample comprises a buffer comprising about 3 to about 4 M guanidinium thiocyanate, about 6% Triton X-100 (v/v), about 5 % DTT (w/w) and about 10 mM citrate monohydrate.

Optionally, a chelating agent can be added to the biological sample. Chelating agents that bind metal ions are of special interest in nucleic acid stability. Many DNases use Zn²⁺ as a cofactor for its activity and the use of a chelating agent inhibits those DNases by withdrawing the cofactor. In one embodiment, ethylenediaminetetraacetic acid (EDTA) and/or ethylene glycol-bis(β-aminoethylether)-N,N,N′,N′-tetraacetic acid (EGTA) is/are used as a chelating agent. In a preferred embodiment, EDTA is used as a chelating agent. The concentration of the chelating agent is preferably at least 1 mM, at least 5 mM, at least 10 mM, at least 50 mM, at least 100 mM, at least 150 mM, at least 180 mM, at least 200 mM, at least 250 mM, at least 400 mM, at least 500 mM or at least 1 M in the biological sample.

To improve the quality and to prevent degradation of nucleic acids, preferably RNA, isolated by means of this invention, “stabilizers” may be also added in certain embodiments of the invention. In one embodiment, ammonium salt(s) and/or sulfate salt(s) are used as stabilizers and added to the fluid test sample or the biological sample. In a preferred embodiment, ammonium sulfate is used as stabilizer and added to the fluid test sample or the biological sample of step (a). In one embodiment, the concentration of the stabilizer is at least 1 mM, at least 5 mM, at least 10 mM, at least 50 mM, at least 70 mM, at least 100 mM, at least 150 mM, at least 180 mM, at least 200 mM, at least 250 mM, at least 500 mM or at least 1 M. In a preferred embodiment, the concentration of the DNA stabilizer is at least 70 mM. In another preferred embodiment, ammonium salt(s) and/or sulfate salt(s), more preferably ammonium sulfate, is/are added to the biological sample of step (a), more preferably to a final concentration of at least 70 mM ammonium sulfate.

In a preferred embodiment of the method of the present invention, said fluid test sample comprises the biological sample, guanidinium thiocyanate as chaotropic agent, dithiothreitol as reducing agent and dihydro sodium citrate or citrate monohydrate. In this embodiment, it is even more preferred that guanidinium thiocyanate is contained in a range from about 40% to about 50% (w/v), and dithiothreitol is contained in a range from about 2% to about 20% (w/w), preferably about 5% to about 15% (w/w), preferably about 5% to about 10% DTT (w/w). In a preferred embodiment of the method of the present invention, said fluid test sample comprises the biological sample, guanidinium thiocyanate as chaotropic agent, Triton X-100 as detergent, dithiothreitol as reducing agent and citrate monohydrate. In this embodiment, it is even more preferred that guanidinium thiocyanate is contained in a range from about 3 M to about 4 M, Triton X-100 is contained in a range from about 3% to about 10% (v/v), preferably at about 6% (v/v), dithiothreitol is contained in a range from about 2.5% to about 15% (w/w), preferably at about 5% (w/w) and citrate monohydrate is contained in a range from about 5 to about 20 mM, preferably at about 10 mM.

In a preferred embodiment of the method of the present invention, said fluid test sample comprises the biological sample, guanidinium thiocyanate as chaotropic agent, Tris-HCl and Triton X-100 as detergent. In this embodiment, it is even more preferred that guanidinium thiocyanate is contained in a range from 3 M to 5 M, even more preferred in a range from 3.5 M to 4.5 M, most preferably about 3.6 M, Tris-HCl is contained in a range from 40 to 60 mM, most preferably 50 mM, and Triton X-100 is contained in a range from 2% to 10% (v/v), most preferably 6% (v/v). In this embodiment, it is most preferred that guanidinium thiocyanate is contained with a concentration of about 3.6 M, Tris-HCl is contained with a concentration of about 50 mM and Triton X-100 is contained with a concentration of about 6% (v/v).

In a preferred embodiment of the method of the present invention, said fluid test sample comprises the biological sample, guanidinium thiocyanate as chaotropic agent, Tris-HCl and Triton X-100 as detergent. In this embodiment, it is even more preferred that guanidinium thiocyanate is contained in a range from 1 M to 4 M, even more preferred in a range from 1.5 M to 4 M, and most preferably at about 3.6 M, Tris-HCl is contained in a range from 40 to 60 mM, most preferably 50 mM, and Triton X-100 is contained in a range from 2% to 10% (w/v), most preferably 6% (w/v). In this embodiment, it is most preferred that guanidinium thiocyanate is contained with a concentration of about 3 to 4 M, Tris-HCl is contained with a concentration of about 50 mM and Triton X-100 is contained with a concentration of about 6% (w/v).

In a preferred embodiment of the method of the present invention, said fluid test sample has a pH of less than 11. It is further preferred for the method of the present invention that the fluid test sample has a pH of about 4 to about 10, more preferably of about 5 to about 9, even more preferably of about 5 to about 8, even more preferably of about 5 to about 7, and most preferably of about 5.8.

In a preferred embodiment of the method of the present invention, said method is conducted at a pH of less than 11. It is further preferred for the method of the present invention that the method is conducted at a pH of about 4 to about 10, more preferably at a pH of about 5 to about 9, even more preferably at a pH of about 5 to about 8, even more preferably at a pH of about 5 to about 7, and most preferably at a pH of about 5.8.

For the method of the present invention, it is preferred that said provision of a fluid test sample comprises the step of contacting a biological sample, preferably a viral sample, with a lysis buffer. Thus, in said embodiment, contacting the biological sample with the lysis buffer creates the fluid test sample. The lysis of a biological sample, like e.g. viral sample, is crucial for the subsequent steps. It is further preferred that the step of contacting a biological sample, preferably a viral sample, with a lysis buffer is followed by or comprises a step of heating the biological sample. It is further preferred that said step of heating the biological sample is at a temperature in the range from about 80° C. to about 95° C., more preferably at a temperature in the range from about 85° C. to about 95° C., even more preferably at a temperature in the range from about 90° C. to about 95° C., and even more preferably at a temperature of about 95° C. It is further preferred that said step of heating the biological sample is conducted before step b) of the method of the present invention. Said step of heating the biological sample is preferably carried out for about 5 to about 15 minutes, more preferably for about 8 to about 12 minutes and even more preferably for about 10 minutes.

In a preferred embodiment, the lysis buffer comprises guanidinium thiocyanate as chaotropic agent, DTT as reducing agent and Triton X-100 as detergent. In this embodiment, it is even more preferred that guanidinium thiocyanate is contained in a range from about 3 M to about 4 M, even more preferred in a range from about 3.5 M to about 4 M, most preferably about 3.6 M, DTT is contained in a range from about 5% to about 15 % (w/w), most preferably about 5% (w/w), and Triton X-100 is contained in a range from about 2% to about 10% (v/v), most preferably about 6% (v/v). In this embodiment, it is most preferred that guanidinium thiocyanate is contained with a concentration of about 3.6 M, DTT is contained with a concentration of about 5% (w/w) and Triton X-100 is contained with a concentration of about 6% (v/v).

The present invention further relates to the use of any of the methods as described herein for detecting a viral infection, more preferably for detecting a viral nucleic acid, even more preferably a viral RNA, even more preferably a viral RNA of Coronaviridae, even more preferably a viral RNA of a SARS-CoV virus and most preferably the viral RNA of SARS-CoV-2. The embodiments of the method of the present invention also apply to the respective use and vice versa.

The present invention also refers to a kit-of-parts comprising a medium for size-exclusion and a size-exclusion chromatography device for isolating the nucleic acid of a fluid test sample, wherein the fluid test sample comprises i) a biological sample, ii) a chaotropic agent with a concentration of at least 1 M in the fluid test sample, and iii) a detergent. Means for carrying out the method for isolating nucleic acids of this invention can be comprised in said kit-of-parts. Such a kit-of-parts may further comprise solutions for the lysis of the biological sample, e.g. lysis buffers as defined above, which may be a chaotropic agent with a concentration of at least 1 M in the fluid test sample. The kit-of-parts may further comprise a solution for precipitation of non-nucleic acid components. In any way, the kit-of-parts comprises a size-exclusion chromatography device for conducting the size-exclusion chromatography and for collecting the respective eluate of the fluid test sample after size-exclusion-chromatography. Preferably, for size-exclusion-chromatography, a resin may be used, wherein the resin is incorporated into a spin column and/or is a size exclusion column. The embodiments of the method of the present invention also apply to the respective kit-of-parts and vice versa.

The present invention also relates to a method for detecting a viral infection, preferably for detecting a viral nucleic acid, more preferably a viral RNA, even more preferably a viral RNA of Coronaviridae, even more preferably a viral RNA of a SARS-CoV virus, most preferably the viral RNA of SARS-CoV-2, wherein said method comprises:
a) provision of a fluid test sample, which comprises
   i) a biological sample,
   ii) a chaotropic agent with a concentration of at least 1 M in the fluid test sample, and
   iii) a detergent,
b) contacting said fluid test sample with a medium for size-exclusion chromatography, and
c) purifying the nucleic acid with size-exclusion chromatography.

The embodiments of the method for isolating a nucleic acid of the present invention also apply to the respective method for detecting a viral infection and vice versa.

In the following, embodiments comprising of different combinations of the above-mentioned obligatory and optional steps for the isolation of the nucleic acids will be presented:

A preferred embodiment of the method of the present invention relates to a method for isolating a nucleic acid, said method comprising:
a) provision of a fluid test sample, which comprises
   i) a biological sample,
   ii) a chaotropic agent with a concentration of at least 1.5 M in the fluid test sample, and
   iii) a detergent,
b) contacting said fluid test sample with a medium for size-exclusion chromatography, and
c) purifying the nucleic acid with size-exclusion chromatography.

A preferred embodiment of the method of the present invention relates to a method for isolating a nucleic acid, said method comprising:
a) provision of a fluid test sample, which comprises
   i) a biological sample,
   ii) a chaotropic agent with a concentration of at least 2 M in the fluid test sample, and
   iii) a detergent,
b) contacting said fluid test sample with a medium for size-exclusion chromatography, and
c) purifying the nucleic acid with size-exclusion chromatography.

In a preferred embodiment, the method of the present invention relates to a method for isolating a nucleic acid, said method comprising:
a) provision of a fluid test sample, which comprises
   i) a biological sample,
   ii) a chaotropic agent with a concentration of at least 2 M in the fluid test sample, wherein the chaotropic agent is guanidinium thiocyanate, and
   iii) a detergent,
b) contacting said fluid test sample with a medium for size-exclusion chromatography, and
c) purifying the nucleic acid with size-exclusion chromatography.

In a preferred embodiment, the method of the present invention relates to a method for isolating a nucleic acid, said method comprising:
a) provision of a fluid test sample, which comprises
   i) a biological sample,
   ii) a chaotropic agent with a concentration of at least 2 M in the fluid test sample, wherein the chaotropic agent is guanidinium thiocyanate, and
   iii) a detergent, wherein the detergent is Triton X-100,
b) contacting said fluid test sample with a medium for size-exclusion chromatography, and
c) purifying the nucleic acid with size-exclusion chromatography.

In a preferred embodiment, the method of the present invention relates to a method for isolating a nucleic acid of a viral RNA, said method comprising:
a) provision of a fluid test sample, which comprises
   i) a biological sample,
   ii) a chaotropic agent with a concentration of at least 2 M in the fluid test sample, wherein the chaotropic agent is guanidinium thiocyanate, and
   iii) a detergent, wherein the detergent is Triton X-100,
b) contacting said fluid test sample with a medium for size-exclusion chromatography, and
c) purifying the nucleic acid with size-exclusion chromatography.

In a preferred embodiment, the method of the present invention relates to a method for isolating a nucleic acid, said method comprising:
a) provision of a fluid test sample, which comprises
   i) a biological sample,
   ii) a chaotropic agent with a concentration of at least 3 M in the fluid test sample, wherein the chaotropic agent is guanidinium thiocyanate, and
   iii) a detergent, wherein the detergent is Triton X-100,
b) contacting said fluid test sample with a medium for size-exclusion chromatography, and
c) purifying the nucleic acid with size-exclusion chromatography.

In a preferred embodiment, the method of the present invention relates to a method for isolating a nucleic acid of a viral RNA, said method comprising:
a) provision of a fluid test sample, which comprises
   i) a biological sample,
   ii) a chaotropic agent with a concentration of at least 3 M in the fluid test sample, wherein the chaotropic agent is guanidinium thiocyanate, and
   iii) a detergent, wherein the detergent is Triton X-100,
b) contacting said fluid test sample with a medium for size-exclusion chromatography, and
c) purifying the nucleic acid with size-exclusion chromatography.

In a preferred embodiment, the method of the present invention relates to a method for isolating a nucleic acid, said method comprising:
a) provision of a fluid test sample, which comprises
   i) a biological sample,
   ii) a chaotropic agent with a concentration of at least 3.5 M in the fluid test sample, wherein the chaotropic agent is guanidinium thiocyanate, and
   iii) a detergent, wherein the detergent is Triton X-100,
b) contacting said fluid test sample with a medium for size-exclusion chromatography, and
c) purifying the nucleic acid with size-exclusion chromatography.

In a preferred embodiment, the method of the present invention relates to a method for isolating a nucleic acid of a viral RNA, said method comprising:
a) provision of a fluid test sample, which comprises
   i) a biological sample,
   ii) a chaotropic agent with a concentration of at least 3.5 M in the fluid test sample, wherein the chaotropic agent is guanidinium thiocyanate, and
   iii) a detergent, wherein the detergent is Triton X-100,
b) contacting said fluid test sample with a medium for size-exclusion chromatography, and
c) purifying the nucleic acid with size-exclusion chromatography.

In a preferred embodiment, the method of the present invention relates to a method for isolating a nucleic acid, said method comprising:
a) provision of a fluid test sample, which comprises
   i) a biological sample,
   ii) a chaotropic agent with a concentration of at least 4 M in the fluid test sample, wherein the chaotropic agent is guanidinium thiocyanate, and
   iii) a detergent, wherein the detergent is Triton X-100,
b) contacting said fluid test sample with a medium for size-exclusion chromatography, and
c) purifying the nucleic acid with size-exclusion chromatography.

In a preferred embodiment, the method of the present invention relates to a method for isolating a nucleic acid of a viral RNA, said method comprising:
a) provision of a fluid test sample, which comprises
   i) a biological sample,
   ii) a chaotropic agent with a concentration of at least 4 M in the fluid test sample, wherein the chaotropic agent is guanidinium thiocyanate, and
   iii) a detergent, wherein the detergent is Triton X-100,
b) contacting said fluid test sample with a medium for size-exclusion chromatography, and
c) purifying the nucleic acid with size-exclusion chromatography.

In a preferred embodiment, the method of the present invention relates to a method for isolating a nucleic acid, said method comprising:
a) provision of a fluid test sample, which comprises
   i) a biological sample,
   ii) a chaotropic agent with a concentration of at least 1 M in the fluid test sample, wherein the chaotropic agent is guanidinium thiocyanate, and
   iii) a detergent, preferably wherein the detergent is Triton X-100 or sodium lauroyl sarcosinate,
      optionally heating the fluid test sample at a temperature in the range of about 80° C. to about 95° C. for about 5 to 15 minutes, more preferably for about 10 minutes;
b) contacting said fluid test sample with a medium for size-exclusion chromatography, and
c) purifying the nucleic acid with size-exclusion chromatography.

In a preferred embodiment, the method of the present invention relates to a method for isolating a nucleic acid, said method comprising:
a) provision of a fluid test sample, which comprises
   i) a biological sample,
   ii) a chaotropic agent with a concentration of at least 1.5 M in the fluid test sample, wherein the chaotropic agent is guanidinium thiocyanate, and
   iii) a detergent, preferably wherein the detergent is Triton X-100 or sodium lauroyl sarcosinate,
      optionally heating the fluid test sample at a temperature in the range of about 80° C. to about 95° C. for about 5 to 15 minutes, more preferably for about 10 minutes;
b) contacting said fluid test sample with a medium for size-exclusion chromatography, and
c) purifying the nucleic acid with size-exclusion chromatography.

In a preferred embodiment, the method of the present invention relates to a method for isolating a nucleic acid, said method comprising:
a) provision of a fluid test sample, which comprises
   i) a biological sample,
   ii) a chaotropic agent with a concentration of at least 2 M in the fluid test sample, wherein the chaotropic agent is guanidinium thiocyanate, and
   iii) a detergent, preferably wherein the detergent is Triton X-100 or sodium lauroyl sarcosinate,
      optionally heating the fluid test sample at a temperature in the range of about 80° C. to about 95° C. for about 5 to 15 minutes, more preferably for about 10 minutes;
b) contacting said fluid test sample with a medium for size-exclusion chromatography, and
c) purifying the nucleic acid with size-exclusion chromatography.

In a preferred embodiment, the method of the present invention relates to a method for isolating a nucleic acid, said method comprising:
a) provision of a fluid test sample, which comprises
   i) a biological sample,
   ii) a chaotropic agent with a concentration of at least 2.5 M in the fluid test sample, wherein the chaotropic agent is guanidinium thiocyanate, and
   iii) a detergent, preferably wherein the detergent is Triton X-100 or sodium lauroyl sarcosinate,
      optionally heating the fluid test sample at a temperature in the range of about 80° C. to about 95° C. for about 5 to 15 minutes, more preferably for about 10 minutes;
b) contacting said fluid test sample with a medium for size-exclusion chromatography, and
c) purifying the nucleic acid with size-exclusion chromatography.

In a preferred embodiment, the method of the present invention relates to a method for isolating a nucleic acid, said method comprising:
a) provision of a fluid test sample, which comprises
   i) a biological sample,
   ii) a chaotropic agent with a concentration of at least 3 M in the fluid test sample, wherein the chaotropic agent is guanidinium thiocyanate, and
   iii) a detergent, preferably wherein the detergent is Triton X-100 or sodium lauroyl sarcosinate,
      optionally heating the fluid test sample at a temperature in the range of about 80° C. to about 95° C. for about 5 to 15 minutes, more preferably for about 10 minutes;
b) contacting said fluid test sample with a medium for size-exclusion chromatography, and
c) purifying the nucleic acid with size-exclusion chromatography.

In a preferred embodiment, the method of the present invention relates to a method for isolating a nucleic acid, said method comprising:
a) provision of a fluid test sample, which comprises
   i) a biological sample,
   ii) a chaotropic agent with a concentration of at least 3.5 M in the fluid test sample, wherein the chaotropic agent is guanidinium thiocyanate, and
   iii) a detergent, preferably wherein the detergent is Triton X-100 or sodium lauroyl sarcosinate,
      optionally heating the fluid test sample at a temperature in the range of about 80° C. to about 95° C. for about 5 to 15 minutes, more preferably for about 10 minutes;
b) contacting said fluid test sample with a medium for size-exclusion chromatography, and
c) purifying the nucleic acid with size-exclusion chromatography.

In a preferred embodiment, the method of the present invention relates to a method for isolating a nucleic acid, said method comprising:
a) provision of a fluid test sample, which comprises
   i) a biological sample,
   ii) a chaotropic agent with a concentration of at least 4 M in the fluid test sample, wherein the chaotropic agent is guanidinium thiocyanate, and
   iii) a detergent, preferably wherein the detergent is Triton X-100 or sodium lauroyl sarcosinate,
      optionally heating the fluid test sample at a temperature in the range of about 80° C. to about 95° C. for about 5 to 15 minutes, more preferably for about 10 minutes;
b) contacting said fluid test sample with a medium for size-exclusion chromatography, and
c) purifying the nucleic acid with size-exclusion chromatography.

In one preferred embodiment, the present invention relates to a method for detecting a viral infection, preferably for detecting a viral nucleic acid, more preferably a viral RNA, even more preferably a viral RNA of Coronaviridae, even more preferably a viral RNA of a SARS-CoV virus, most preferably the viral RNA of SARS-CoV-2, wherein said method comprises:
a) provision of a fluid test sample, which comprises
   i) a biological sample,
   ii) a chaotropic agent with a concentration of at least 1.5 M in the fluid test sample, and
   iii) a detergent, preferably wherein the detergent is Triton X-100 or sodium lauroyl sarcosinate,
b) contacting said fluid test sample with a medium for size-exclusion chromatography, and
c) purifying the nucleic acid with size-exclusion chromatography.

In one preferred embodiment, the present invention relates to a method for detecting a viral infection, preferably for detecting a viral nucleic acid, more preferably a viral RNA, even more preferably a viral RNA of Coronaviridae, even more preferably a viral RNA of a SARS-CoV virus, most preferably the viral RNA of SARS-CoV-2, wherein said method comprises:
a) provision of a fluid test sample, which comprises
   i) a biological sample,
   ii) a chaotropic agent with a concentration of at least 2 M in the fluid test sample, and
   iii) a detergent, preferably wherein the detergent is Triton X-100 or sodium lauroyl sarcosinate,
b) contacting said fluid test sample with a medium for size-exclusion chromatography, and
c) purifying the nucleic acid with size-exclusion chromatography.

In one preferred embodiment, the present invention relates to a method for detecting a viral infection, preferably for detecting a viral nucleic acid, more preferably a viral RNA, even more preferably a viral RNA of Coronaviridae, even more preferably a viral RNA of a SARS-CoV virus, most preferably the viral RNA of SARS-CoV-2, wherein said method comprises:
a) provision of a fluid test sample, which comprises
   i) a biological sample,
   ii) a chaotropic agent with a concentration of at least 2 M in the fluid test sample, wherein the chaotropic agent is guanidinium thiocyanate, and
   iii) a detergent, wherein the detergent is Triton X-100,
b) contacting said fluid test sample with a medium for size-exclusion chromatography, and
c) purifying the nucleic acid with size-exclusion chromatography.

In one preferred embodiment, the present invention relates to a method for detecting a viral infection, preferably for detecting a viral nucleic acid, more preferably a viral RNA, even more preferably a viral RNA of Coronaviridae, even more preferably a viral RNA of a SARS-CoV virus, most preferably the viral RNA of SARS-CoV-2, wherein said method comprises:
a) provision of a fluid test sample, which comprises
   i) a biological sample,
   ii) a chaotropic agent with a concentration of at least 2 M in the fluid test sample, wherein the chaotropic agent is guanidinium thiocyanate, and
   iii) a detergent, wherein the detergent is Triton X-100,
b) contacting said fluid test sample with a medium for size-exclusion chromatography, and
c) purifying the nucleic acid with size-exclusion chromatography.

In one preferred embodiment, the present invention relates to a method for detecting a viral infection, preferably for detecting a viral nucleic acid, more preferably a viral RNA, even more preferably a viral RNA of Coronaviridae, even more preferably a viral RNA of a SARS-CoV virus, most preferably the viral RNA of SARS-CoV-2, wherein said method comprises:
a) provision of a fluid test sample, which comprises
   i) a biological sample,
   ii) a chaotropic agent with a concentration of at least 3 M in the fluid test sample, wherein the chaotropic agent is guanidinium thiocyanate, and
   iii) a detergent, wherein the detergent is Triton X-100,
b) contacting said fluid test sample with a medium for size-exclusion chromatography, and
c) purifying the nucleic acid with size-exclusion chromatography.

In one preferred embodiment, the present invention relates to a method for detecting a viral infection, preferably for detecting a viral nucleic acid, more preferably a viral RNA, even more preferably a viral RNA of Coronaviridae, even more preferably a viral RNA of a SARS-CoV virus, most preferably the viral RNA of SARS-CoV-2, wherein said method comprises:
a) provision of a fluid test sample, which comprises
   i) a biological sample,
   ii) a chaotropic agent with a concentration of at least 3 M in the fluid test sample, wherein the chaotropic agent is guanidinium thiocyanate, and
   iii) a detergent, wherein the detergent is Triton X-100,
b) contacting said fluid test sample with a medium for size-exclusion chromatography, and
c) purifying the nucleic acid with size-exclusion chromatography.

In one preferred embodiment, the present invention relates to a method for detecting a viral infection, preferably for detecting a viral nucleic acid, more preferably a viral RNA, even more preferably a viral RNA of Coronaviridae, even more preferably a viral RNA of a SARS-CoV virus, most preferably the viral RNA of SARS-CoV-2, wherein said method comprises:
a) provision of a fluid test sample, which comprises
   i) a biological sample,
   ii) a chaotropic agent with a concentration of at least 3.5 M in the fluid test sample, wherein the chaotropic agent is guanidinium thiocyanate, and
   iii) a detergent, wherein the detergent is Triton X-100,
b) contacting said fluid test sample with a medium for size-exclusion chromatography, and
c) purifying the nucleic acid with size-exclusion chromatography.

In one preferred embodiment, the present invention relates to a method for detecting a viral infection, preferably for detecting a viral nucleic acid, more preferably a viral RNA, even more preferably a viral RNA of Coronaviridae, even more preferably a viral RNA of a SARS-CoV virus, most preferably the viral RNA of SARS-CoV-2, wherein said method comprises:
a) provision of a fluid test sample, which comprises
   i) a biological sample,
   ii) a chaotropic agent with a concentration of at least 4 M in the fluid test sample, wherein the chaotropic agent is guanidinium thiocyanate, and
   iii) a detergent, wherein the detergent is Triton X-100,
b) contacting said fluid test sample with a medium for size-exclusion chromatography, and
c) purifying the nucleic acid with size-exclusion chromatography.

In one preferred embodiment, the present invention relates to a method for detecting a viral infection, preferably for detecting a viral nucleic acid, more preferably a viral RNA, even more preferably a viral RNA of Coronaviridae, even more preferably a viral RNA of a SARS-CoV virus, most preferably the viral RNA of SARS-CoV-2, wherein said method comprises:
a) provision of a fluid test sample, which comprises
   i) a biological sample,
   ii) a chaotropic agent with a concentration of at least 1 M in the fluid test sample, wherein the chaotropic agent is guanidinium thiocyanate, and
   iii) a detergent, preferably wherein the detergent is Triton X-100 or sodium lauroyl sarcosinate,
      optionally heating the fluid test sample at a temperature in the range of about 80° C. to about 95° C. for about 5 to 15 minutes, more preferably for about 10 minutes;
b) contacting said fluid test sample with a medium for size-exclusion chromatography, and
c) purifying the nucleic acid with size-exclusion chromatography.

In one preferred embodiment, the present invention relates to a method for detecting a viral infection, preferably for detecting a viral nucleic acid, more preferably a viral RNA, even more preferably a viral RNA of Coronaviridae, even more preferably a viral RNA of a SARS-CoV virus, most preferably the viral RNA of SARS-CoV-2, wherein said method comprises:
a) provision of a fluid test sample, which comprises
   i) a biological sample,
   ii) a chaotropic agent with a concentration of at least 1.5 M in the fluid test sample, wherein the chaotropic agent is guanidinium thiocyanate, and
   iii) a detergent, preferably wherein the detergent is Triton X-100 or sodium lauroyl sarcosinate,
      optionally heating the fluid test sample at a temperature in the range of about 80° C. to about 95° C. for about 5 to 15 minutes, more preferably for about 10 minutes;
b) contacting said fluid test sample with a medium for size-exclusion chromatography, and
c) purifying the nucleic acid with size-exclusion chromatography.

In one preferred embodiment, the present invention relates to a method for detecting a viral infection, preferably for detecting a viral nucleic acid, more preferably a viral RNA, even more preferably a viral RNA of Coronaviridae, even more preferably a viral RNA of a SARS-CoV virus, most preferably the viral RNA of SARS-CoV-2, wherein said method comprises:
a) provision of a fluid test sample, which comprises
   i) a biological sample,
   ii) a chaotropic agent with a concentration of at least 2 M in the fluid test sample, wherein the chaotropic agent is guanidinium thiocyanate, and
   iii) a detergent, preferably wherein the detergent is Triton X-100 or sodium lauroyl sarcosinate,
      optionally heating the fluid test sample at a temperature in the range of about 80° C. to about 95° C. for about 5 to 15 minutes, more preferably for about 10 minutes;
b) contacting said fluid test sample with a medium for size-exclusion chromatography, and
c) purifying the nucleic acid with size-exclusion chromatography.

In one preferred embodiment, the present invention relates to a method for detecting a viral infection, preferably for detecting a viral nucleic acid, more preferably a viral RNA, even more preferably a viral RNA of Coronaviridae, even more preferably a viral RNA of a SARS-CoV virus, most preferably the viral RNA of SARS-CoV-2, wherein said method comprises:
a) provision of a fluid test sample, which comprises
   i) a biological sample,
   ii) a chaotropic agent with a concentration of at least 2.5 M in the fluid test sample, wherein the chaotropic agent is guanidinium thiocyanate, and
   iii) a detergent, preferably wherein the detergent is Triton X-100 or sodium lauroyl sarcosinate,
      optionally heating the fluid test sample at a temperature in the range of about 80° C. to about 95° C. for about 5 to 15 minutes, more preferably for about 10 minutes;
b) contacting said fluid test sample with a medium for size-exclusion chromatography, and
c) purifying the nucleic acid with size-exclusion chromatography.

In one preferred embodiment, the present invention relates to a method for detecting a viral infection, preferably for detecting a viral nucleic acid, more preferably a viral RNA, even more preferably a viral RNA of Coronaviridae, even more preferably a viral RNA of a SARS-CoV virus, most preferably the viral RNA of SARS-CoV-2, wherein said method comprises:
a) provision of a fluid test sample, which comprises
   i) a biological sample,
   ii) a chaotropic agent with a concentration of at least 3 M in the fluid test sample, wherein the chaotropic agent is guanidinium thiocyanate, and
   iii) a detergent, preferably wherein the detergent is Triton X-100 or sodium lauroyl sarcosinate,
      optionally heating the fluid test sample at a temperature in the range of about 80° C. to about 95° C. for about 5 to 15 minutes, more preferably for about 10 minutes;
b) contacting said fluid test sample with a medium for size-exclusion chromatography, and
c) purifying the nucleic acid with size-exclusion chromatography.

In one preferred embodiment, the present invention relates to a method for detecting a viral infection, preferably for detecting a viral nucleic acid, more preferably a viral RNA, even more preferably a viral RNA of Coronaviridae, even more preferably a viral RNA of a SARS-CoV virus, most preferably the viral RNA of SARS-CoV-2, wherein said method comprises:
a) provision of a fluid test sample, which comprises
   i) a biological sample,
   ii) a chaotropic agent with a concentration of at least 3.5 M in the fluid test sample, wherein the chaotropic agent is guanidinium thiocyanate, and
   iii) a detergent, preferably wherein the detergent is Triton X-100 or sodium lauroyl sarcosinate,
      optionally heating the fluid test sample at a temperature in the range of about 80° C. to about 95° C. for about 5 to 15 minutes, more preferably for about 10 minutes;
b) contacting said fluid test sample with a medium for size-exclusion chromatography, and
c) purifying the nucleic acid with size-exclusion chromatography.

In one preferred embodiment, the present invention relates to a method for detecting a viral infection, preferably for detecting a viral nucleic acid, more preferably a viral RNA, even more preferably a viral RNA of Coronaviridae, even more preferably a viral RNA of a SARS-CoV virus, most preferably the viral RNA of SARS-CoV-2, wherein said method comprises:
a) provision of a fluid test sample, which comprises
   i) a biological sample,
   ii) a chaotropic agent with a concentration of at least 4 M in the fluid test sample, wherein the chaotropic agent is guanidinium thiocyanate, and
   iii) a detergent, preferably wherein the detergent is Triton X-100 or sodium lauroyl sarcosinate,
      optionally heating the fluid test sample at a temperature in the range of about 80° C. to about 95° C. for about 5 to 15 minutes, more preferably for about 10 minutes;
b) contacting said fluid test sample with a medium for size-exclusion chromatography, and
c) purifying the nucleic acid with size-exclusion chromatography.

It is further preferred for all preferred embodiments mentioned above that the fluid test sample further comprises a reducing agent, preferably DTT or TCEP, more preferably DTT, even more preferably about 5% to about 15% DTT (w/w), and even more preferably about 5% DTT (w/w).

It must be noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

When used herein, the term “about” is understood to mean that there can be variation in the respective value or range (such as pH, concentration, percentage, molarity, number of amino acids, time etc.) that can be up to 5 %, up to 10 % of the given value. For example, if a formulation comprises about 5 mg/ml of a compound, this is understood to mean that a formulation can have between 4.5 and 5.5 mg/ml.

All publications and patents cited in this disclosure are incorporated by reference in their entirety. To the extent, the material incorporated by reference contradicts or is inconsistent with this specification, the specification will supersede any such material.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

Throughout this specification and the claims which follow, unless the context requires otherwise, the word “comprise”, and variations such as “comprises” and “comprising”, will be understood to imply the inclusion of a stated integer or step or group of integers or steps, but not the exclusion of any other integer or step or group of integer or step. When used herein the term “comprising” can be substituted with the term “containing” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

In each instance herein, any of the terms “comprising”, “consisting essentially of” and “consisting of” may be replaced with either of the other two terms.

Several documents are cited throughout the text of this specification. Each of the documents cited herein (including all patents, patent applications, scientific publications, manufacturer’s specifications, instructions, etc.), whether supra or infra, are hereby incorporated by reference in their entirety. Nothing herein is to be construed as an admission that the invention is not entitled to antedate such disclosure by virtue of prior invention.

The invention is further characterized by the following items:

1. A method for isolating a nucleic acid, said method comprising:
a) provision of a fluid test sample, which comprises
   i) a biological sample,
   ii) a chaotropic agent with a concentration of at least 1 M in the fluid test sample, and
   iii) a detergent,
b) contacting said fluid test sample with a medium for size-exclusion chromatography, and
c) purifying the nucleic acid with size-exclusion chromatography.

2. The method of item 1, wherein the nucleic acid of step c) is directly applied to PCR, RT-PCR or NGS.

3. The method of item 1 or 2, which is conducted without the addition of protease.

4. The method of any one of the preceding items, which is conducted without a bind-wash-elute-step.

5. The method of any one of the preceding items, wherein the chaotropic agent has a concentration of at least 2 M in the fluid test sample, preferably of at least 2.5 M in the fluid test sample, more preferably of at least 3 M in the fluid test sample.

6. The method of any one of the preceding items, wherein the medium for size exclusion is a resin used for size-exclusion chromatography (SEC); preferably hydroxylated methacrylic polymers or a cross-linked dextrane; more preferably a dextrane cross-linked with N,N′-methylenebisacrylamide; a water-based mobile phase, such as water, an aqueous organic solvent or an aqueous buffer/solution mobile phase.

7. The method of any one of the preceding items, wherein the chaotropic agent is guanidinium thiocyanate or guanidinium hydrochloride, preferably guanidinium thiocyanate.

8. The method of any one of the preceding items, wherein said nucleic acid is RNA and/or DNA, preferably RNA.

9. The method of any one of the preceding items, wherein said RNA is a viral RNA, preferably a viral RNA of Coronaviridae, more preferably a viral RNA of a SARS-CoV virus, most preferably the viral RNA of SARS-CoV-2.

10. The method of any one of the preceding items, wherein the biological sample is a viral sample, a fecal sample, a saliva sample, a sputum sample, a mouth swab sample, a throat swab sample or a nasal swab sample.

11. The method of any one of the preceding items, wherein said fluid test sample further comprises EDTA, polidocanol, DTT, dihydro sodium citrate, or a buffering substance, preferably Tris-HCl.

12. The method of any one of the preceding items, wherein said provision of a fluid test sample comprises the step of contacting a biological sample, preferably a viral sample, with a lysis buffer.

13. Use of any of the methods according to items 1 to 12 for detecting a viral infection, preferably for detecting a viral nucleic acid, more preferably a viral RNA, even more preferably a viral RNA of Coronaviridae, even more preferably a viral RNA of a SARS-CoV virus, most preferably the viral RNA of SARS-CoV-2.

14. Kit-of-parts comprising a medium for size-exclusion and a size-exclusion chromatography device for isolating a nucleic acid of a fluid test sample, wherein the fluid test sample comprises i) a biological sample, ii) a chaotropic agent with a concentration of at least 1 M in the fluid test sample, and iii) a detergent.

15. A method for detecting a viral infection, preferably for detecting a viral nucleic acid, more preferably a viral RNA, even more preferably a viral RNA of Coronaviridae, even more preferably a viral RNA of a SARS-CoV virus, most preferably the viral RNA of SARS-CoV-2, said method comprising:
a) provision of a fluid test sample, which comprises
   i) a biological sample,
   ii) a chaotropic agent with a concentration of at least 1 M in the fluid test sample, and
   iii) a detergent,
b) contacting said fluid test sample with a medium for size-exclusion chromatography, and
c) purifying the nucleic acid with size-exclusion chromatography.

### EXAMPLES OF THE PRESENT INVENTION

### Materials and Methods

Described herein are materials needed for nucleic acid extraction and a general method for the isolation of nucleic acids. Changes indicated in the Examples supersede the general method. This general method is suitable to obtain high-purity nucleic acid from all common cultured cell lines such as HeLa, COS or HEK and also primary cells from eukaryotic origin. The purified RNA or DNA can comprise particularly long fragments, is free of contaminants and enzyme inhibitors like chaotropic reagents and organic solvents and is highly suitable for all downstream applications, like PCR or RT-PCR.

### Materials

This list shows materials needed for isolating nucleic acids:
Microcentrifuge with rotor for 1.5 ml and 2 ml reaction tubes set to 650 g;
Vortex mixer;
One reaction tube (1.5 ml) per sample for the lysis step, preferably safe-lock;
One reaction tube (2 ml) per sample for column preparation;
One reaction tube (1.5 ml) per sample for elution and collection of the purified genomic DNA;
Pipets for 10 µl and up to 200 µl, corresponding pipet tips;
Size exclusion chromatography column: Spin columns filled with respective amounts of Sephacryl S100, S200, S300, S400, S500; or the Toyopearl resins see above;
Lysis buffer: see the respective Example;

### Isolation of Nucleic Acids

In the following, a method for isolating viral DNA or RNA is described. It consists of a one-step-method for reverse clearing of virus DNA or RNA out of chaotropic solutions, in which virus particles are contained.

This method allows lysis WITHOUT an explicit lysis step via enzymatic digestion and is extremely potent and advantageous for the depletion of inhibitory substances such as chaotropic agents. Though the chaotropic agent is needed to allow the lysis of the virus particle to be able to gain the viral DNA or RNA, it is at the same time beneficial and extremely necessary to deplete the chaotropic agent by e.g. size-exclusion chromatography as described herein as much as possible as otherwise a PCR or RT-PCR as subsequent step would be prevented by the inhibitory effect of the chaotropic agent.

### Example 1

Fluid test sample: Provided SWAB sample with viral particle of SARS-CoV-2 is rinsed with buffer containing
   43% (w/v) guanidinium thiocyanate (GITC) (3.64 M),
   5% (w/v) polydocanol,
   2% (w/v) dithiothreitol and
   dihydro sodium citrate (in the range of 20-50 mM);
   this buffer has beforehand been diluted with PBS (1 Vol. of fluid test sample and 0.9 Vol. PBS);
   NO incubation or separate lysis or temperature-step;
   the initial SWAB-sample can be provided as a dry SWAB sample, in not chaotropic transport
   medium or already in chaotropic transport medium;
Column preparation (during incubation): If air is present in the column, remove by vortexing;
   Centrifuge 1 min at 1000 x g; place the column in a 1.5 ml tube;
Clearing and purification: Add 100 µl of fluid test sample vertically through cap, pipet slowly into the column, or pipet slowly on resin bed; centrifuge 1 min at 1000 x g;
The eluate contains the isolated DNA/ RNA and can be further processed by e.g. SARS-CoV-2 specific PCR-detection. Resulting RNA was analyzed by agarose gel electrophoresis.

### Example 2

Fluid test sample: Provided SWAB sample with viral particle of SARS-CoV-2 is rinsed with buffer containing
   4 M guanidinium thiocyanate (GITC; NO diluting of the original GITC-concentration, because even
   4 M GITC is depleted enough by the size exclusion chromatography),
   50 mM Tris-HCl and
   3% (v/v) Triton X-100;
   NO incubation or separate lysis or temperature-step;
   the initial SWAB-sample can be provided as a dry SWAB sample, in not chaotropic transport
   medium or already in chaotropic transport medium;
Column preparation (during incubation): If air is present in the column, remove by vortexing;
   Centrifuge 1 min at 1000 x g; place the column in a 1.5 ml tube;
Clearing and purification: Add 100 µl of fluid test sample vertically through cap, pipet slowly into the column, or pipet slowly on resin bed; Centrifuge 1 min at 1000 x g;
The eluate contains the isolated DNA/ RNA and can be further processed by e.g. SARS-CoV-2 specific PCR-detection. Resulting RNA was analyzed by agarose gel electrophoresis.

### Example 3

Fluid test sample: Provided SWAB sample with viral particle of SARS-CoV-2 is rinsed with buffer containing
   2 M guanidinium thiocyanate (GITC),
   50 mM Tris-HCl and
   3% (v/v) Triton X-100;
   NO incubation or separate lysis or temperature-step;
   the initial SWAB-sample can be provided as a dry SWAB sample, in not chaotropic transport
   medium or already in chaotropic transport medium;
Column preparation (during incubation): If air is present in the column, remove by vortexing;
   Centrifuge 1 min at 1000 x g; place the column in a 1.5 ml tube;
Clearing and purification: Add 100 µl of fluid test sample vertically through cap, pipet slowly into the column, or pipet slowly on resin bed; Centrifuge 1 min at 1000 x g;
The eluate contains the isolated DNA/ RNA and can be further processed by e.g. SARS-CoV-2 specific PCR-detection. Resulting RNA was analyzed by agarose gel electrophoresis.

### Example 4 A More Detailed Description of the Column Preparation Is Described

Place a Spin Column into a 1.5 ml reaction tube. Note: If air is present in the column: remove by vortexing briefly. Snap off the bottom closure of the Spin Column. Important: Loosen the screw cap of the spin column a half turn to avoid yield reduction due to generation of a vacuum. Place back the column into the 2 ml collection tube.
Centrifuge for 1 minute at 1000 g. See section materials and equipment needed above for details. Discard the 2 ml reaction tube containing the flow through.
Place the prepared spin column into a new 1.5 ml reaction tube for elution of the sample and both together in a rack. Continue with “Purification” (below).

### Example 5 A More Detailed Description of the Purification Is Described

Fluid test sample as described above was transferred to the prepared column from Example 4 as described.
Open cap and pipet the lysed sample slowly onto the middle of the resin bed of the prepared (Spin) Column of Example 4. Close cap. Important: Loosen the screw cap of the spin column a half turn to avoid yield reduction.
Centrifuge 1 minute at 1000 g. The purified genomic RNA flows through the column into the 1.5 ml elution tube. Discard the spin column.
The eluted genomic DNA can be used immediately or stored at 4° C. or -20° C.
For the purification, microplates of the 96-type may be used.

### Example 6: Conducting PCR

PCR was conducted in alternative 1 with cobas z480 system. The detection for SARS-CoV-2 was done in channel 465-510 (FAM) respectively for beta-corona-viruses in the channel 540-580 (HEX). As amplification-control, the reagents of a buffer negative control kit was used and the SARS-CoV-2 positive control kit (from Roche). The detection of the also prepared and amplified internal controls (IC) was done in channel 680-700 (Cy5.5).

PCR was conducted in alternative 2 with Anchor SARS-CoV-2 PCR Test. The detection of SARS-CoV-2 and beta-corona-viruses was done with separated detection-systems, but in the same channel 465-510 (FAM), while the also prepared internal controls like in alternative 1 as described above were detected in channel 540-580 (HEX).

### Example 7: Showing Depletion of Chaotropic Agent GITC

The following experiments show how the chaotropic agent GITC (2-4 M) is depleted by following the method of the present invention.

### Experiment 1 of Example 7

The inventors have tested, which filter matrix (G50, S200 or S400) used in the size-exclusion chromatography of the methods of the present invention and in which amount (650 µl and 800 µl) was most suitable for depleting guanidinium thiocyanate (GITC) in a concentration of 43 % (w/w) in A. Dest. Additionally, various volumes of the fluid test sample have been tested. Therefore, two different filter-plates (Agilent Seahorse) have been manufactured.

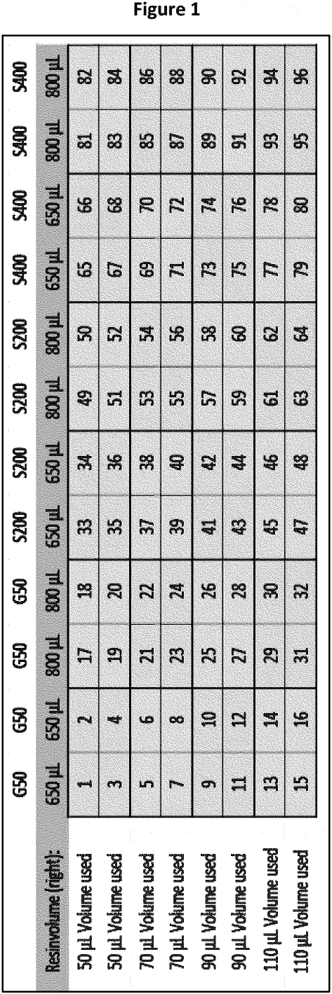

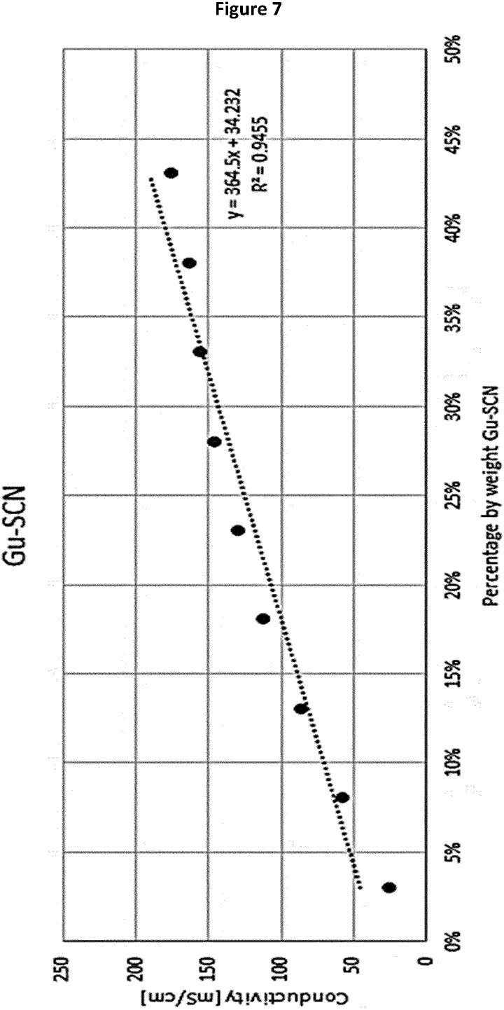

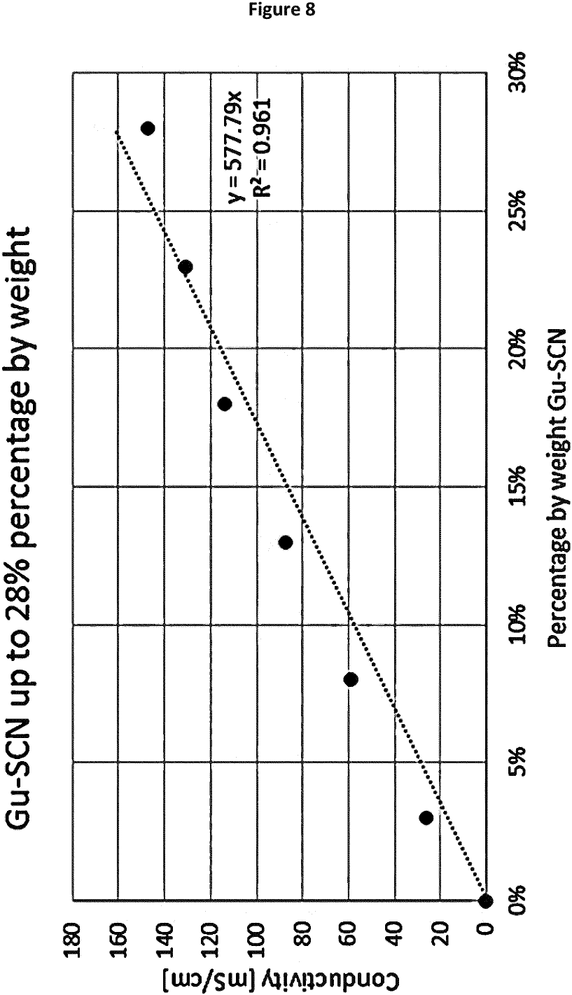

Plate 1: In FIG. 1, the respective volumes of the fluid test sample are given. 10 µl of the respective eluates was diluted 1 : 100 each and the conductivities were measured. Standard-curve was established according to the FIGS. 7 and 8.

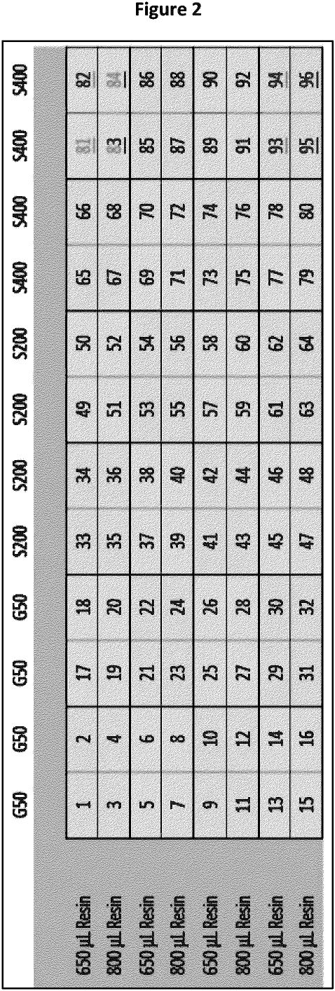

Plate 2: The remaining eluate was processed by a second plate 2 (same arrangement as for the plate 1, shown in FIG. 2), wherein plate 2 contained various amounts of S400 (650 µl or 800 µl resin). Some eluates from the plate 2 (see underlinements) were then tested in an inhibition-PCR (1, 4 and 8 µl, Sensifast PCR with Hox target).

### Conductivity of the Eluate From Plate 1

The conductivity of 43 % guanidinium thiocyanat was 188 mS, while the eluates of S400 showed an average in conductivity of 1.3 mS. The eluates of S200 showed in average 1.6 mS, while G50 showed an average value of conductivity of 30 mS. The conductivity-measurements clearly showed that G50-eluates compared to the sephacryl-eluates showed a clearly higher conductivity, which is a proof for a not so good depletion of GITC. Between S200 and S400 the differences were not so enormous, however, for S400 an even better depletion of GITC could be shown.

### Inhibition-PCR of Eluates From Plate 2

The following eluates from plate 2 were tested (overview according to Table 1 given below):

TABLE 1

showing processed eluates in PCR

| Sample No. | Volume used | plate 1 resin | plate 2 resin |
|---|---|---|---|
| 81 | 50 µL | S400 800 µL | S400 650 µL |
| 82 | 50 µL | S400 800 µL | S400 650 µL |
| 83 | 50 µL | S400 800 µL | S400 650 µL |
| 84 | 50 µL | S400 800 µL | S400 650 µL |
| 93 | 110 µL | S400 800 µL | S400 650 µL |
| 94 | 110 µL | S400 800 µL | S400 650 µL |
| 95 | 110 μL | S400 800 µL | S400 650 µL |
| 96 | 110 μL | S400 800 µL | S400 650 µL |

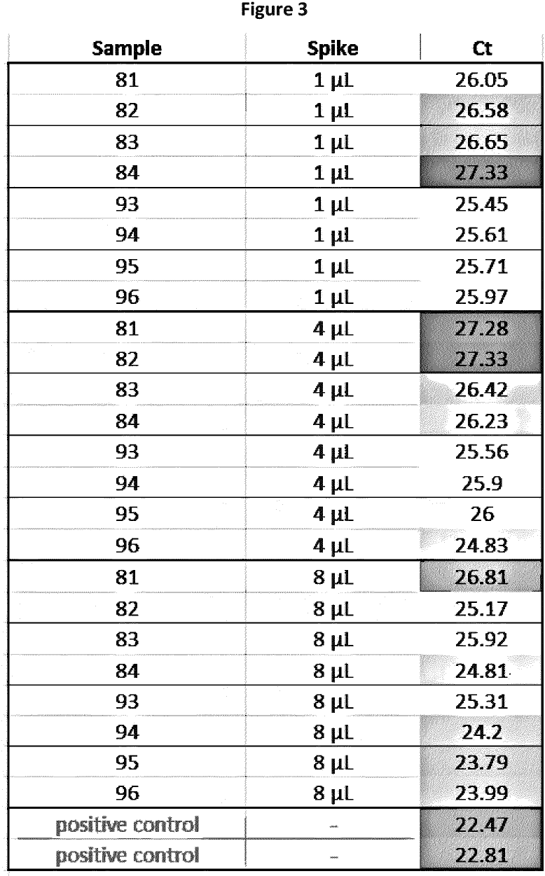

For all PCR-reactions, the same amount of DNA-template was present. The positive control had 8 µl A. dest., while the other samples each had a spike (substitution of the water-content of 1, 4 or 8 µl). The results thereof were shown in FIG. 3.

Sample 95 and 96 showed the best amplification (best Ct) of all samples. An amount of 800 µl S400 in both clearing plates and a volume of 110 µl of the fluid test sample showed the best depletion of GITC. The better Ct-values of the samples 93, 94, 95 and 96 could be due to the less EDTA-influence from the column. The higher the volume put into the column the less is the eluate diluted by the void-volume (TE buffer) of the column and the less EDTA-containing is the eluate. EDTA is a known inhibitor of PCR. At the same time, Ct-values were in a tendency worse for samples with a less spike-amount.

The experiments showed a sufficient depletion of GITC for an amplification by TAQ-polymerase.

### Experiment 2 of Example 7

6 samples according to the following Table 2 were prepared:

TABLE 2

| Sample | Buffer | Column purification | First elution | Second Elution |
|---|---|---|---|---|
| 1 | 100 µLRoche | Yes | 4 µLPCR | 1 und 7.4 µLPCR |
| 2 | 100 µLRoche | Yes | 4 µL PCR | 1 und 7.4 µL PCR |
| 3 | 100 µL TE | Yes | 4 µL PCR | 1 und 7.4 µL PCR |
| 4 | 100 µL TE | Yes | 4 µL PCR | 1 und 7.4 µLPCR |
| 5 | 100 µL TE | No | - | 1 und 7.4 µLPCR |
| 6 | 100 µLTE | No | - | 1 und 7.4 µLPCR |

All samples contained an identical RNA spike (PEDV), which could be detected by RT-qPCR. Additionally, all samples contained a plasmid-concentration of 5 ng/ µL (PCMV-β) as “carrier-DNA”. Samples 1-4 were processed via plate 1 (S400 - 800 µl resin) and the first eluate was spiked into PCR (4 µl). The remaining eluate was again processed via plate 2 and the eluates thereof were also spiked into PCR (1 and 7.4 µl). At the same time, samples 5 and 6 were not processed via columns, but were directly spiked into PCR (1 and 7.4 µl).

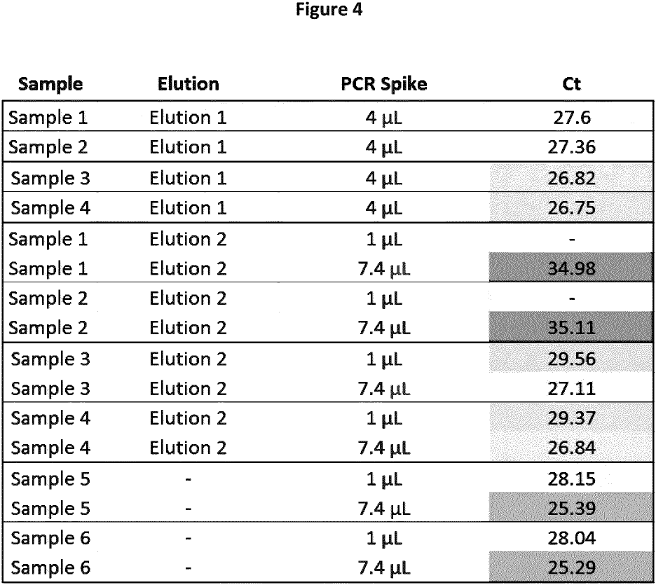

The results of this procedure are given in FIG. 4 and are as follows: The first eluate of the fluid test sample according to Example 1 as given above was well amplifiable, with only a slight inhibition compared to the TE-containing samples (Delta Ct about 0.8 - 1). The second elutions for the probes with a fluid test sample according to Example 1 were completely inhibited, while the second elution of the TE-buffer was not inhibited. Not inhibited were the samples 5 and 6, which were directly spiked into PCR. No Taq-Polymerase was used here.

### Experiment 3 of Example 7

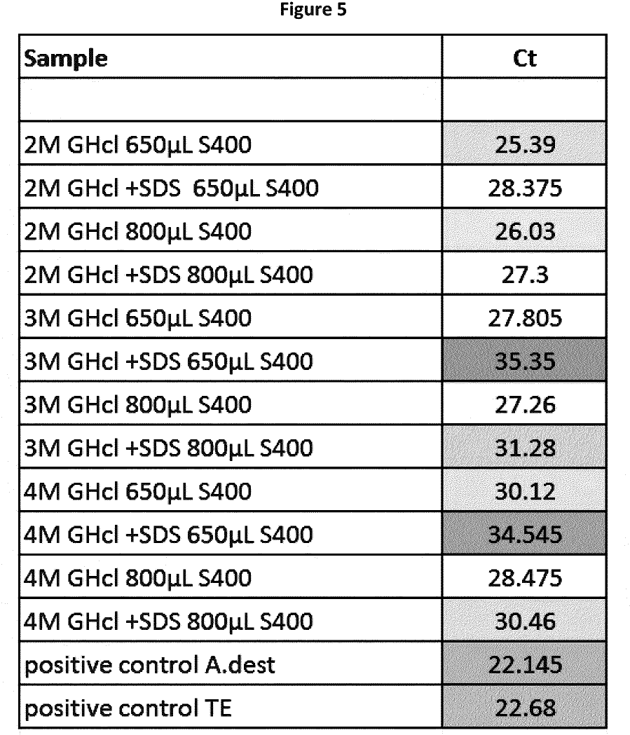

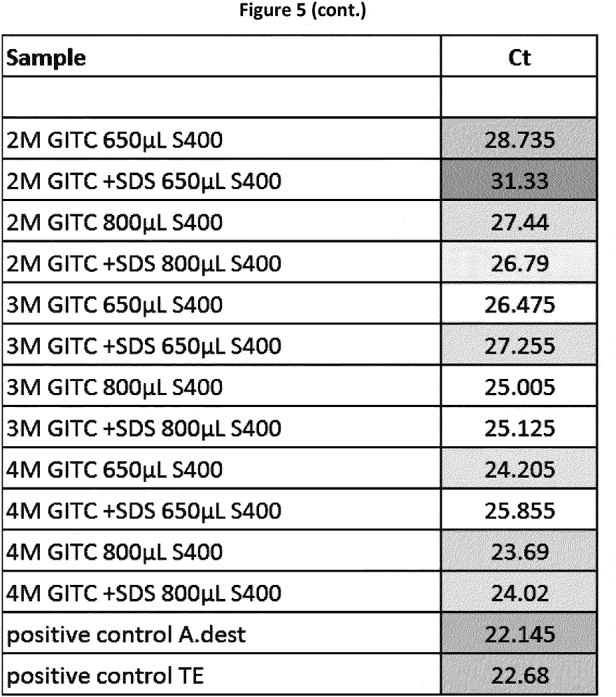

96er plates were prepared with each 800 µl and 650 µl S400. At the same time 6 different buffer were prepared with guanidinium thiocyanate and guanidinium-hydrochloride with a molarity of each 2 M, 3 M and 4 M. From each buffer, 100 µl was processed over the plate (each over 800 µl and 650 µl S400). In an additional variant, after pipetting 100 µl buffer (6 different ones) additionally 30 µl 0.3 M SDS-solution was additionally applied to achieve precipitation of the chaotropic salt on the column. 8 µl of the thereby gained eluates was spiked in a HOX PCR (DNA already contained in PCR-Mix; Sensifast TAQ PCR Mix used). In each case, double-samples were used and the average values were used for the results. Results are given in FIG. 5.

Results: Guanidinium-hydrochloride was more inhibited at higher concentrations. For the samples with guanidinium-hydrochloride, the additional SDS had clearly a disadvantageous effect. For GITC, SDS had no or a very small negative effect on the CT. For both chaotropic salts is a higher resin-volume (800 µl) advantageous for the Ct. TE buffer in the positive control changes the Ct very slightly (shift back), however, the inhibitory effect is very minimal.

### Experiment 4 of Example 7

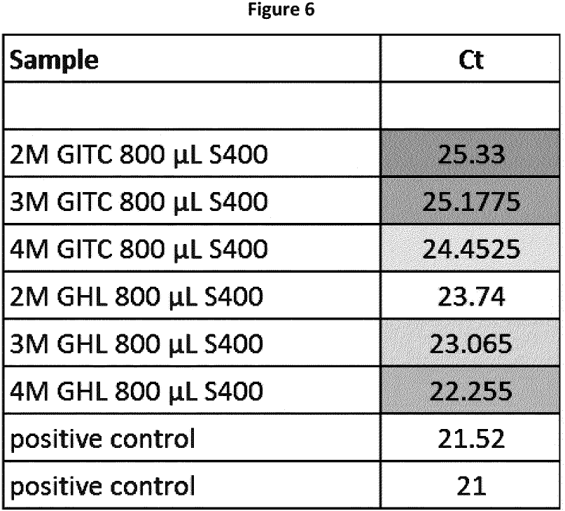

Experiment 3 was repeated with 6 buffers. However, only 800 µl S400 was tested and no SDS was added. For each buffer-variant, 4 samples were processed and used in PCR. Each Ct in the results is thus an average value of 4 samples. The positive-control was Aqua Dest. and DNA. The result thereof is shown in FIG. 6. Guanidinium-hydrochloride has a less strong inhibitory effect on the PCR.

### Example 8: General Results

In total, 94 samples (plus NC and PC) were analyzed in both amplification systems. Buffers with 40% GHCl all showed precipitations or very late values for IC-refinding (50/68) and at the positive SARS-CoV-2 samples unacceptable late IC-values or no finding of positive samples (10/35).

The diluted buffer with in total 23 % GITC according to Example 1 showed instead for all samples a refinding of ICs with expected IC-values (31/31) and a refinding of the target with expected IC-values compared to routine-diagnostic (9/11). Only slightly positive samples (Ct > 35) were not detected in positive-control samples. The samples of both PCR-alternatives according to Example 6 showed identical or almost identical values.

The buffer according to Example 1, containing 43 % GITC 1:1 in PBS, used for rinsing the SWAB samples lead to isolation of SARS-CoV-2 RNA and the refinding of the together with the sample applied ICs. Samples rinsed with 40 % GHCl showed no refinding of targets and additionally a worse refinding of ICs.

The method of the present invention further allows the use of a 4- to 5-times less sample volume compared to methods of the state of the art (100 µl compared to 400 µl or 500 µl). The methods of the present invention are in summary extremely advantageous for the manual high-troughput for testing on SARS-CoV-2.

### Example 9

### Aim of This Experiment

The aim of this experiment was to test, which size exclusion filter matrix in which amount is best suited to desalt a watery solution containing a high concentration of the chaotropic salt guanidinium thiocyanate. In addition, different column loading volumes of the samples were tested.

### Experimental Description

After filling the resins into the filter plate, excess water, in which the filter materials (resins) were re-suspended, was first removed by centrifugation at 1000 g. Then, the sample (3.6 M guanidinium thiocyanate in A. dest) was loaded and the filter plate was again centrifuged at 1000 g. The resulting eluates were collected and the conductivity of the eluates was measured.

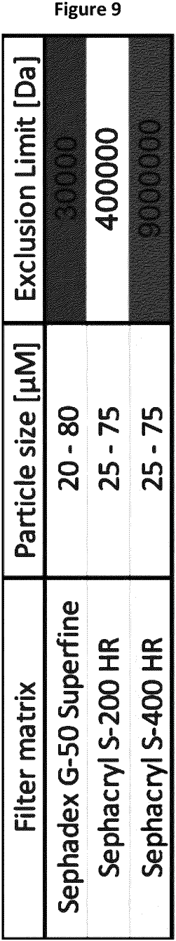

The filter matrices as shown in FIG. 9 were tested in this experiment:

For example, Sephadex G-50 Superfine (recommended for group separation, e.g. desalting) was used. According to https://www.cytivalifesciences.com/en/us/shop/chromatography/resins/size-exclusion/sephadex-g-50-superfine-p-05487: “Sephadex G-50 Superfine is a well-established gel filtration resin for desalting and buffer exchange of biomolecules > 30 000 molecular weight. The Superfine’s small bead size give higher efficiency.”

Further, Sephacryl S-200 HR (recommended for high-resolution fractionation) was used: According to https://www.cytivalifesciences.com/en/us/shop/chromatography/resins/size-exclusion/sephacryl-s-200-hr-p-05621: “Sephacryl High Resolution size exclusion chromatography resins allow fast and reproducible purification of proteins, polysaccharides, and other macromolecules by size exclusion chromatography at laboratory and industrial scale.”

Additionally, Sephacryl S-400 HR (recommended for high-resolution fractionation) was used. According to https://www.cytivalifesciences.com/en/us/shop/chromatography/resins/size-exclusion/sephacryl-s-400-hr-p-06271: “Sephacryl High Resolution size exclusion chromatography resins allow fast and reproducible purification of proteins, polysaccharides, and other macromolecules by size exclusion chromatography at laboratory and industrial scale.”

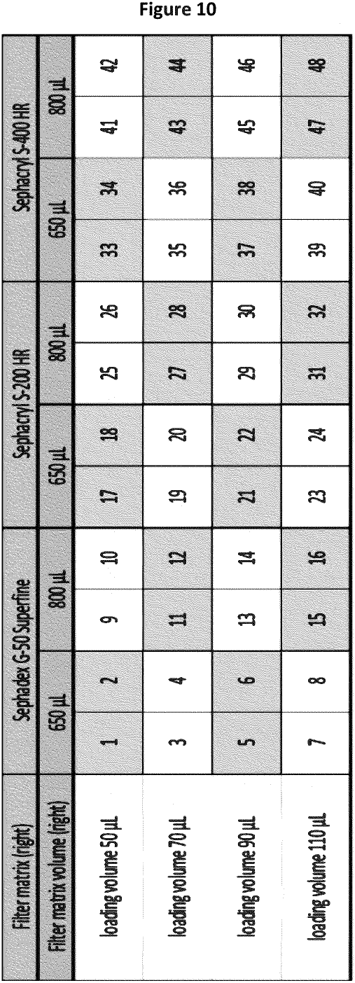

The arrangement of the different experimental parameters on the filter plate is shown in FIG. 10.

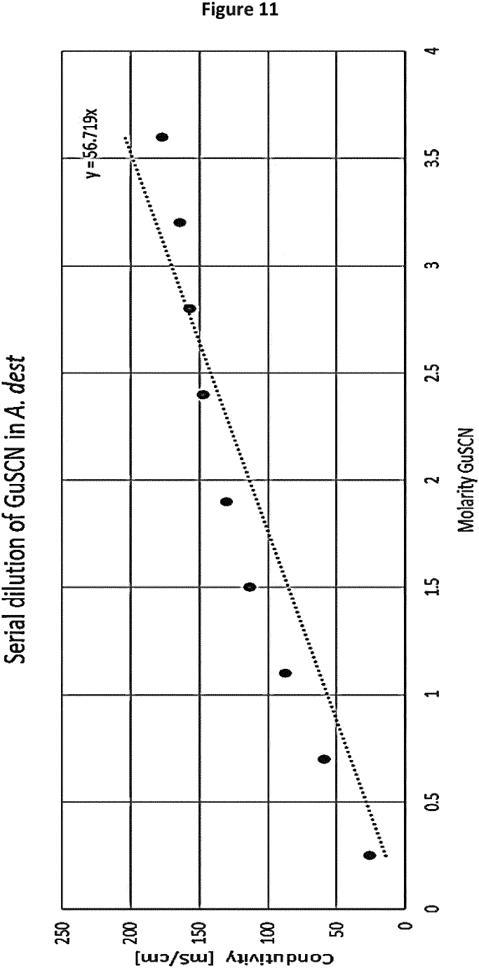

In order to calculate the residual content of GuSCN from the measured conductivity of the eluates, a dilution series with different concentrations of GuSCN was created (see FIG. 11).

### Results

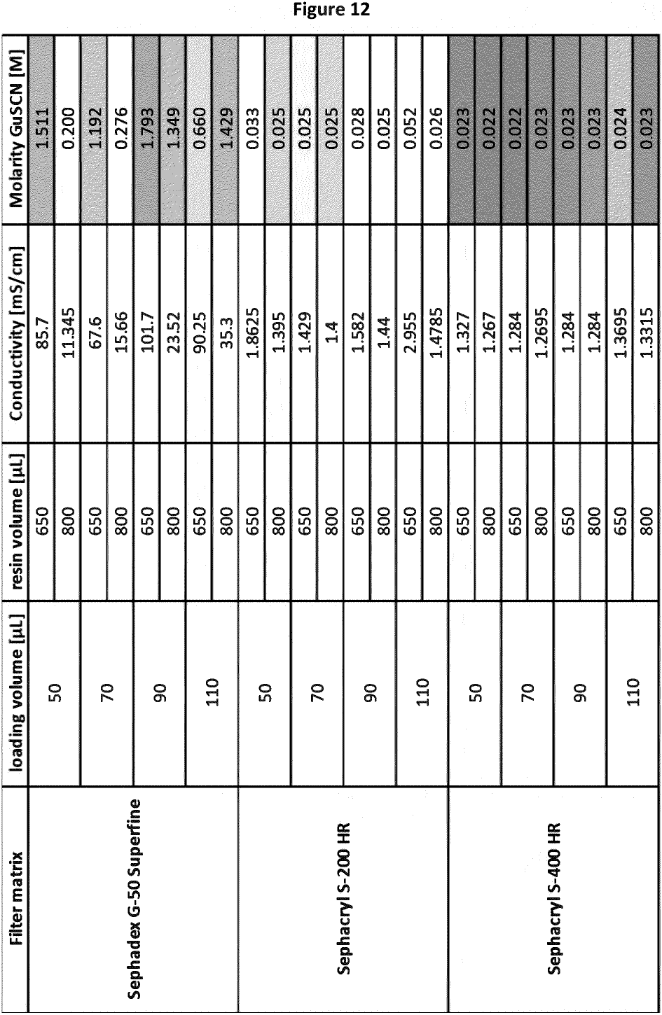

The results of the serial dilution series were used to calculate the molarity of the collected eluates, as can be seen in FIG. 12 (each value is the average of 2 replicates).

Very surprisingly, it turned out that the filter material specially recommended by the manufacturer for desalting (Sephadex G-50) is not the best at removing the chaotropic salt GuSCN using centrifugation-based filtration. Instead, the other two filter materials tested (Sephacryl S-400 HR and Sephacryl S-200 HR) showed the highest depletion capacity for GuSCN, although they are intended for high-resolution fractionation of high-molecular biomolecules by the manufacturer.

It turned out, that the eluates from the samples processed via Sephacryl S-400 HR were even below the concentration given in the literature (see https://www.mn-net.com/media/pdf/02/1a/74/Instruction-NucleoSpin-Gel-and-PCR-Clean-up.pdf), above which a PCR is inhibited.

One explanation for the unusual behaviour is the high flow rate of the mobile phase during the purification (centrifugation). Using filter matrices with small pores (like Sephadex G-50), the salts may not have time to enter the pores in significant quantities, whereas a large-pored material ensures this even at high flow rates.

It was not initially expected that a centrifugation-based filtration with an SEC filter material, which according to the manufacturer’s product information is used to separate macromolecules from each other, would remove small molecules such as salts much more efficiently than materials marketed specifically for salt removal and buffer exchange. This use of filter materials intended for high-resolution fractionation (e.g. Sephacryl S-400 HR) as a desalting material for chaotropic salts in combination with the purification of RNA/DNA molecules by centrifugation-driven filtration could surprisingly be discovered by the present invention.

### Example 10

### Aim of the Experiment

In Example 9 above, it was successfully shown that a highly chaotropic solution can be desalinated extremely well with the help of SEC materials, resulting in a salt concentration in the eluate that does not inhibit a PCR.

This experiment of Example 10 shows that high concentrations of chaotropic salts are required to protect nucleic acids in the lysate and that nucleic acids in the sample being released during lysis can subsequently be successfully isolated from all other buffer components. The successful isolation of nucleic acid in the eluate was then confirmed in a PCR.

### Experimental Description

Six variations of typical state of the art lysis buffer used in nucleic acid isolations containing different concentrations of the chaotropic salt guanidinium thiocyanate (GuSCN) were produced. Furthermore, all six lysis buffers did contain identical concentrations of 6 % [v/v] Triton X-100, 5 % [w/w] dithiothreitol (reducing agent) and 10 mM sodium citrate dihydrate (pH buffer). All buffers were adjusted to a pH of 5.8 using NaOH. Additionally, a transport buffer (containing the biological sample - e.g. viruses or bacteria) was produced, containing 0.9 % NaCl and 50 mM TRIS, adjusted to pH 7.5. To simulate a real biological origin, a cotton swab was used to introduce saliva of a real person into the transport media. As biological sample (viroid), an encapsulated RNA fragment was used (Internal Control RNA/IC RNA from RIDA^{®}GENE SARS-CoV-2 PCR Kit).

The final lysate was then mixed at room temperature and comprised 50 µL lysis buffer (six different buffers), 50 µL of the transport buffer and 20 µL of IC RNA. No incubation was performed.

800 µL of Sephacryl S-400 HR suspension were filled into each well of a 96-well filter plate. Excess water, in which the filter material was resuspended was removed by centrifugation at 1000 g. Then, 90 µL of the previously prepared lysate mixtures were loaded and the filter plate was again centrifuged at 1000 g. The eluate was collected and a RT-PCR targeting the introduced IC RNA using the RIDA^{®}GENE SARS-CoV-2 PCR Kit* was performed. PCR sample input was 5 µL (total PCR volume 25 µL). (see https://clinical.r-biopharm.com/wp-content/uploads/sites/3/2020/06/pg6820_ridagene_sars_cov-2_2020-10-28_de_final.pdf)

### Results

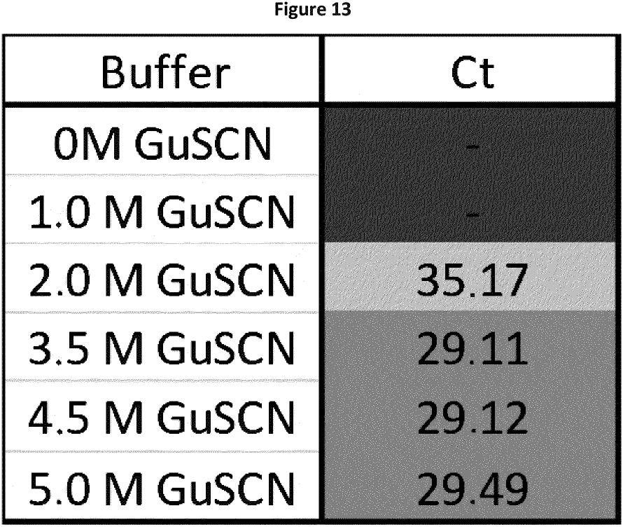

The Ct-values obtained in Example 10 are shown in FIG. 13.

The obtained Ct-values (see FIG. 13) proof that nucleic acid (RNA) was successfully isolated from the lysate and that the obtained eluates are free of inhibiting chaotropic salts and suitable for PCR analysis. The filtration has thus successfully retained the chaotropic salts and has allowed the nucleic acid to pass. Furthermore, lower concentrations than 3.5 M GuSCN in the lysis buffer are not able to protect the nucleic acid (IC RNA) in the sample from degradation by saliva-related ribonucleases. Therefore, concentrations of at least > 1 M, but preferably > 3.5 M GuSCN, in the lysis buffer are necessary in order to enable a loss-free RNA Isolation.

### Example 11

### Aim of the Experiment

Examples 9 and 10 showed that filter materials from the manufacturer Cytiva (Sephadex, Sephacryl) with larger pore sizes surprisingly ensure better salt depletion than the small-pored materials intended for this purpose by the manufacturer.

In this experiment of Example 11, this desalting performance is now to be compared with filter materials from another manufacturer (SEC WorkBeads from the company Bioworks) in order to confirm the statement of the Examples presented above.

### Experiment Description

A typical state of the art lysis buffer used in nucleic acid isolations containing 3.6 M of guanidinium thiocyanate (GuSCN) was produced. Furthermore, the lysis buffers did contain identical concentrations of 6 % [v/v] Triton X-100, 5 % [w/w] dithiothreitol (reducing agent) and 10 mM sodium citrate dihydrate (pH buffer). The buffer was adjusted to a pH of 5.8 using NaOH.

In contrast to Example 10, this time the commercially available transport buffer Copan eSwab was used. As biological sample (viroid), an encapsulated RNA fragment was used (Internal Control RNA/IC RNA from RIDA^{®}GENE SARS-CoV-2 PCR Kit).

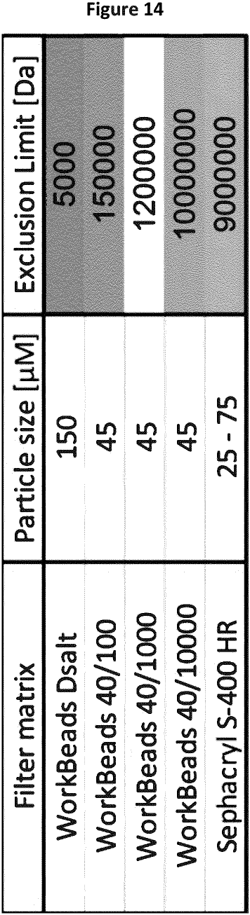

One lysate (lysate 1) was then mixed at room temperature and comprised 50 µL lysis buffer, 50 µL of the transport buffer and 20 µL of IC RNA. No incubation was performed. Additionally, a second type of lysate (lysate 2) was mixed, containing 100 µL lysis buffer and 20 µL of IC RNA. Both lysate variants were processed trough the columns containing the filter materials as can be seen in FIG. 14.

800 µL of the listed filter materials (see FIG. 14) were filled into each well of a 96-well filter plate. Excess water, in which the filter material was re-suspended, was removed by centrifugation at 1000 g. Then 90 µL of the two previously prepared lysate mixtures were loaded and the filter plate was again centrifuged at 1000 g. The eluate was collected and a RT-PCR targeting the introduced IC RNA using the RIDA^{®}GENE SARS-CoV-2 PCR Kit* was performed. PCR sample input was 5 µL (total PCR volume 25 µL).

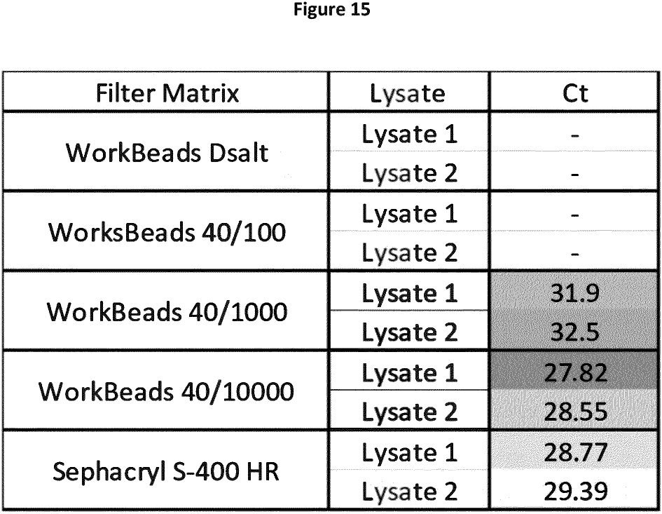

Results: The Ct-values obtained in Example 11 are shown in FIG. 15.

The results of Example 11 confirm the results presented in Examples 9 and 10. Here, too, a better PCR (better Ct-value) was achieved with increasing pore size (size exclusion limit - see FIG. 14): WorkBeads 40/10000 > Sephacryl S-400 HR > WorkBeads 40/1000 > Works Beads 40/100 and WorkBeads Dsalt.

In particular, the WorkBeads 40/10000 show even a better result than Sephacryl S-400 HR (Delta Ct of 1). The reason for this is very likely the even higher pore size (size exclusion limit) of the Workbeads 40/10000.

### Example 12

### Aim of the Experiment

This experiment shows that the invention can indeed be used to successfully purify virus RNA and can be used for subsequent qRT-PCR analysis. For this purpose, inactivated virus were diluted in a common transport medium (Copan eSwab - a very frequently used transport medium for mouth swab samples in human diagnostics) in different virus concentrations into which saliva was previously introduced by 3 different persons via a mouth swab. The transport medium (sample) was then purified using the method as described herein and the eluates were analyzed in a qRT-PCR application for the detection of SARS CoV 2.

### Experiment Description

Initially, saliva (containing naturally occurring ribonucleases) was introduced into separate transport media vessels (Copan eSwab) by 3 people via a mouth swab to simulate clinical samples. Subsequently, different concentrations (100,000 - 1,000 copies / mL) of viruses from a concentrated virus solution (inactivated SARS CoV 2 viral particles - 10,000,000 copies / mL) were introduced into the transport media. This prepared transport medium constituted the sample.

The lysis buffer used in this Example contained 3.6 M of guanidinium thiocyanate as chaotropic salt agent, 6% [v/v] Triton X-100 as detergent, 5% [w/w] dithiothreitol as reducing agent and 10 mM sodium citrate dihydrate (pH buffer). The buffer was adjusted to a pH of 5.8 using NaOH.

50 µL of each sample was then contacted and mixed with 50 µL of said lysis buffer at room temperature to prepare the lysate mixture. No incubation step was performed.

For further processing of the samples, 800 µL of Sephacryl S-400 HR suspension were filled into each well of a 96-well filter plate. Excess water, in which the filter material was resuspended, was removed by centrifugation at 1000 g. Then, 90 µL of the previously prepared lysate mixtures were loaded and the filter plate was again centrifuged at 1000 g.

The eluate was collected and a RT-PCR, targeting the introduced viral RNA using the RIDA^{®}GENE SARS-CoV-2 PCR Kit from R-Biopharm (see https://clinical.r-biopharm.com/wp-content/uploads/sites/3/2020/06/pg6820_ridagene_sars_cov-2_2020-10-28_de_final.pdf) was performed. Each sample was extracted and measured in two technical replicates. The PCR sample input was 5 µL (total PCR volume 25 µL).

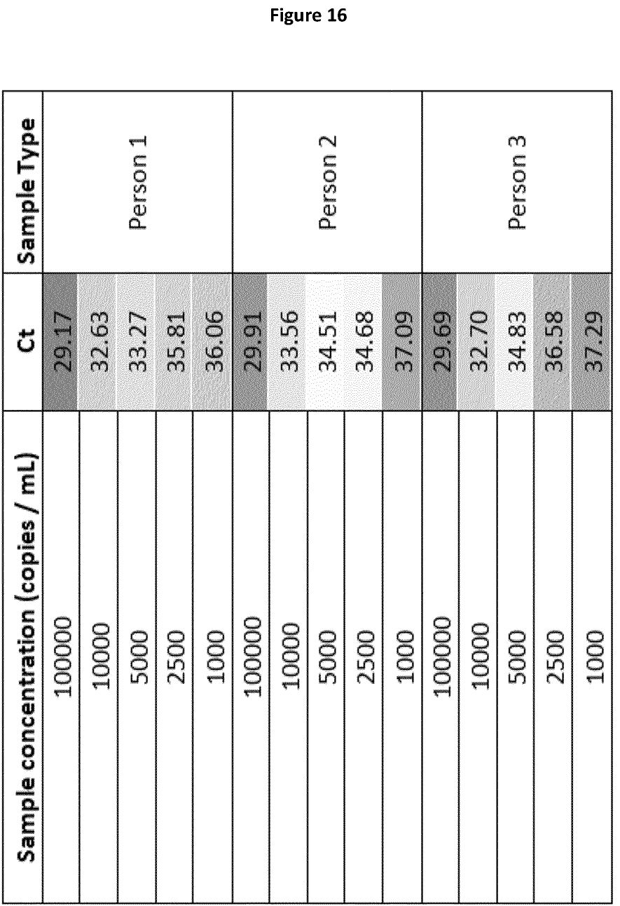

Results: The Ct-values, obtained in Example 12, are shown in FIG. 16.

The obtained Ct-values of this Example 12 (see FIG. 16) proof, that nucleic acid (RNA) was successfully isolated from the prepared SARS-CoV-2-containing samples and that the obtained eluates were free of inhibiting chaotropic salts and were suitable for PCR analysis. The lysis buffer has thus successfully released the viral RNA, has protected the viral RNA. Additionally, the filtration has successfully retained the PCR inhibiting lysis buffer components (e.g., chaotropic salts) and has allowed the viral RNA to pass through. Furthermore, it can be shown that the Ct-values obtained in the qRT-PCR were comparable between the 3 individuals/ persons. All different virus concentrations (100,000 to 1,000 copies/mL) in the prepared samples were successfully detected in the qRT-PCR analysis.

1. A method for isolating a nucleic acid, said method comprising:
a) provision of a fluid test sample, which comprises
i) a biological sample,
ii) a chaotropic agent with a concentration of at least 1 M in the fluid test sample, and
iii) a detergent,
b) contacting said fluid test sample with a medium for size-exclusion chromatography, and
c) purifying the nucleic acid with size-exclusion chromatography.

2. The method of claim 1, wherein the nucleic acid of step c) is directly applied to PCR, RT-PCR or NGS.

3. The method of claim 1 or 2, which is conducted without the addition of protease.

4. The method of any one of the preceding claims, which is conducted without a bind-wash-elute-step.

5. The method of any one of the preceding claims, wherein the chaotropic agent has a concentration of at least 1.5 M in the fluid test sample, preferably of at least 2 M in the fluid test sample, more preferably of at least 2.5 M in the fluid test sample, more preferably of at least 3 M in the fluid test sample, and even more preferably of at least 3.5 M in the fluid test sample.

6. The method of any one of the preceding claims, wherein the medium for size exclusion is a resin used for size-exclusion chromatography (SEC); preferably a hydroxylated methacrylic polymer, a cross-linked dextrane or a cross-linked agarose; more preferably a dextrane cross-linked with N,N′-methylenebisacrylamide; a water-based mobile phase, such as water, an aqueous organic solvent or an aqueous buffer/solution mobile phase.

7. The method of any one of the preceding claims, wherein the chaotropic agent is guanidinium thiocyanate or guanidinium hydrochloride, preferably guanidinium thiocyanate.

8. The method of any one of the preceding claims, wherein the detergent is a non-ionic detergent, preferably Triton, more preferably Triton X-100, or wherein the detergent is a salt of lauroyl sarcosinate, preferably sodium lauroyl sarcosinate, or a derivative thereof.

9. The method of any one of the preceding claims, wherein said nucleic acid is RNA and/or DNA, preferably RNA.

10. The method of any one of the preceding claims, wherein said RNA is a viral RNA, preferably a viral RNA of Coronaviridae, more preferably a viral RNA of a SARS-CoV virus, most preferably the viral RNA of SARS-CoV-2.

11. The method of any one of the preceding claims, wherein the biological sample is a viral sample, a fecal sample, a saliva sample, a sputum sample, a mouth swab sample, a throat swab sample or a nasal swab sample.

12. The method of any one of the preceding claims, wherein said fluid test sample further comprises EDTA, Triton X-100, DTT, citrate monohydrate, dihydro sodium citrate, or a buffering substance, preferably Tris-HCl.

13. The method of any one of the preceding claims, wherein said fluid test sample further comprises a reducing agent, preferably DTT, TCEP or a derivative thereof.

14. The method of any one of the preceding claims, wherein the method further comprises a step of heating the fluid test sample, preferably at a temperature in the range from about 80° C. to about 95° C., preferably before step b).

15. The method of any one of the preceding claims, wherein said provision of a fluid test sample comprises the step of contacting a biological sample, preferably a viral sample, with a lysis buffer.

16. Use of any one of the methods according to claims 1 to 15 for detecting a viral infection, preferably for detecting a viral nucleic acid, more preferably a viral RNA, even more preferably a viral RNA of Coronaviridae, even more preferably a viral RNA of a SARS-CoV virus, most preferably the viral RNA of SARS-CoV-2.

17. Kit-of-parts comprising a medium for size-exclusion and a size-exclusion chromatography device for isolating a nucleic acid of a fluid test sample, wherein the fluid test sample comprises i) a biological sample, ii) a chaotropic agent with a concentration of at least 1 M in the fluid test sample, and iii) a detergent.

18. A method for detecting a viral infection, preferably for detecting a viral nucleic acid, more preferably a viral RNA, even more preferably a viral RNA of Coronaviridae, even more preferably a viral RNA of a SARS-CoV virus, most preferably the viral RNA of SARS-CoV-2, said method comprising:
a) provision of a fluid test sample, which comprises
i) a biological sample,
ii) a chaotropic agent with a concentration of at least 1 M in the fluid test sample, and
iii) a detergent,
b) contacting said fluid test sample with a medium for size-exclusion chromatography, and
c) purifying the nucleic acid with size-exclusion chromatography.
